(19) United States
(12) Patent Application Publication
(10) Pub. No.: US 2010/0078607 A1
(43) Pub. Date: Apr. 1, 2010

(54) NON-RESONANT TWO-PHOTON ABSORPTION RECORDING MATERIAL AND NON-RESONANT TWO-PHOTON ABSORPTION COMPOUND

(75) Inventors: Masaharu AKIBA, Ashigarakami-gun (JP); Eri TAKAHASHI, Ashigarakami-gun (JP); Hiroo TAKIZAWA, Ashigarakami-gun (JP); Hidehiro MOCHIZUKI, Odawara-shi (JP); Hirokazu HASHIMOTO, Odawara-shi (JP)

Correspondence Address: SUGHRUE-265550, 2100 PENNSYLVANIA AVE. NW, WASHINGTON, DC, 20037-3213, US

(73) Assignee: FUJIFILM CORPORATION, Tokyo (JP)

(21) Appl. No.: 12/570,187

(22) Filed: Sep. 30, 2009

(30) Foreign Application Priority Data
Sep. 30, 2008 (JP) 2008-255808

(51) Int. Cl. G02F 1/361; C07C 255/50

(52) U.S. Cl. 252/582; 558/415

(57) ABSTRACT

There is provided a non-resonant two-photon absorption recording material including (a) a non-resonant two-photon absorption compound; and (b) a recording component capable of changing at least either one of a refractive index and a fluorescent intensity. The non-resonant two-photon absorption compound (a) is represented by the following formula (1):

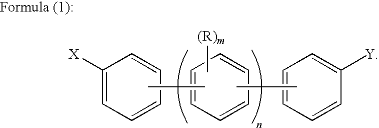

In the formula (1), both of X and Y represent a substituent having a Hammett sigma para value (σp value) of 0 or more and they may be the same as or different from each other; n represents an integer of 1 to 4; R represents a substituent and each R may be the same as or different from every other R; and m represents an integer of 0 to 4.

### BACKGROUND OF THE INVENTION

1. Field of the Invention

The present invention relates to a non-resonant two-photon absorption recording material and a non-resonant two-photon absorption compound. More specifically, the present invention relates to a recording material making it possible to three-dimensionally record a recording pit in the inside of a recording medium by using non-resonant two-photon absorption and read out the recording pit and enabling non-resonant two-photon absorption recording using recording light in a wavelength region shorter than 700 nm, and a two-photon absorption compound.

2. Description of the Related Art

In general, the non-linear optical effect indicates a non-linear optical response proportional to the square, cube or higher power of photoelectric field applied. Known examples of the second-order non-linear optical effect proportional to the square of photoelectric field applied include second harmonic generation (SHG), optical rectification, photorefractive effect, Pockels effect, parametric amplification, parametric oscillation, light sum frequency mixing and light difference frequency mixing. Also, examples of the third-order non-linear optical effect proportional to the cube of photoelectric filed applied include third harmonic generation (THG), optical Kerr effect, self-induced refractive index change and two-photon absorption.

As for the non-linear optical material exhibiting these non-linear optical effects, a large number of inorganic materials have been heretofore found. However, an inorganic material can be hardly used in practice because a so-called molecular design so as to optimize the desired non-linear optical characteristics or various properties necessary for the production of a device is difficult. On the other hand, an organic compound can realize not only optimization of the desired non-linear optical characteristics by the molecular design but also control of other various properties and therefore, the probability of its practical use is high. Thus, an organic compound is attracting attention as a promising non-linear optical material.

In recent years, among non-linear optical characteristics of the organic compound, third-order non-linear optical effects, particularly, non-resonant two-photon absorption, are being taken notice of. The two-photon absorption is a phenomenon of a compound being excited by simultaneously absorbing two photons. In the case where the two-photon absorption occurs in the energy region having no (linear) absorption band of the compound, this is called non-resonant two-photon absorption. In the following, even when not particularly specified, “two-photon absorption” indicates “non-resonant two-photon absorption”. Also, “simultaneous two-photon absorption” is sometimes simply referred to as “two-photon absorption” by omitting “simultaneous”.

Meanwhile, the non-resonant two-photon absorption efficiency is proportional to the square of photoelectric field applied (quadratic dependency of two-photon absorption). Therefore, when a laser is irradiated on a two-dimensional plane, two-photon absorption takes place only in the position having a high electric field strength in the center part of laser spot and utterly no two-photon absorption occurs in the portion having a weak electric field strength in the periphery. On the other hand, in a three-dimensional space, two-photon absorption occurs only in the region having a large electric field strength at the focus where the laser rays are converged through a lens, and two-photon absorption does not take place at all in the off-focus region because the electric field strength is weak. Compared with the linear absorption where excitation occurs in all positions proportionally to the strength of photoelectric field applied, in the non-resonant two-photon absorption, excitation occurs only at one point inside the space because of the quadratic dependency and therefore, the spatial resolution is remarkably enhanced.

Usually, in the case of inducing non-resonant two-photon absorption, a short pulsed laser in the near infrared region having a wavelength longer than the wavelength region where the (linear) absorption band of a compound is present, and not having the absorption of the compound is used in many cases. Thanks to use of near infrared light in a so-called transparent region, the excitation light can reach the inside of a sample without being absorbed or scattered and one point inside the sample can be excited with very high spatial resolution because of the quadratic dependency of non-resonant two-photon absorption.

The present inventors have filed various patent applications relating to a two-photon sensitization-type three-dimensional recording material using a compound capable of inducing non-resonant two-photon absorption. This recording material is a recording material containing at least (1) a two-photon absorption compound (two-photon sensitizer) and (2) a refractive index-modulating material or a fluorescent intensity-modulating material, where (1) efficiently undergoes two-photon absorption and the obtained energy is transferred to (2) by photoexcited electron transfer or energy transfer to change the refractive index or fluorescent intensity of (2), thereby performing the recording. Thanks to use of non-resonant two-photon absorption but not one-photon absorption employed in the process of light absorption of normal optical recording, a recording pit with three-dimensional spatial resolution can be written at an arbitrary position inside of a recording material.

For example, JP-A-2007-87532 (the term “JP-A” as used herein means an “unexamined published Japanese patent application”) discloses a technique using, as (2) a refractive index- or fluorescent intensity-modulating material, a material capable of causing a refractive index modulation by color-forming a dye, or a material capable of causing a fluorescence modulation from non-fluorescence to fluorescent emission or from fluorescent emission to non-fluorescence (a material that creates a refractive index or fluorescence modulation through color formation of a dye or a fluorescent dye). Also, JP-A-2005-320502 discloses a technique using, as (2) a refractive index- or fluorescent intensity-modulating material, a material capable of forming a seed (latent image speck) through very slight color formation of a dye or change of fluorescence and then performing recording and amplification under light irradiation or heating (a refractive index/fluorescence modulation and latent image amplification system; a material that forms a latent image capable of creating a refractive index/fluorescence modulation through color formation of a dye). Furthermore, for example, JP-A-2005-29725 discloses a technique using, as (2) a refractive index-modulating material, a material capable of forming a high-molecular polymer by polymerization and thereby modulating the refractive index (a material that creates a refractive index modulation through polymerization), and JP-A-2005-97538 discloses a technique using, as a refractive index-modulating material, a material capable of forming a very fine polymerized latent image speck and then driving the polymerization (a refractive index modulation and latent image polymerization system; a material that forms a latent image capable of creating a refractive index modulation through polymerization).

In all of these two-photon sensitization-type three-dimensional recording materials described in JP-A-2007-87532, JP-A-2005-320502, JP-A-2005-29725 and JP-A-2005-97538, the material capable of performing two-photon absorption with light of 700 nm or more is used as (1) a two-photon absorption compound (two-photon sensitizer). However, there are various demands in recent years, and above all, for obtaining a higher recording density, a recording material capable of performing non-resonant two-photon absorption recording by using recording light in a wavelength region shorter than 700 nm so as to form a smaller pit in the recording material is required.

Incidentally, Y. Morel, O. Stephan, C. Andraud, and P. L. Baldeck, Synth. Met., 2001, 124, 237 discloses that a compound having a structure shown below exhibits non-resonant two-photon absorption properties with light at 450 to 600 nm.

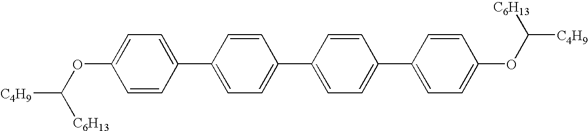

However, the two-photon absorption cross-sectional area of the compound above is about 15 GM at a maximum and even when such a compound is used for the two-photon sensitization-type three-dimensional recording material, fully satisfactory results cannot be expected.

### SUMMARY OF THE INVENTION

An object of the present invention is to provide a two-photon absorption recording material capable of performing non-resonant two-photon absorption recording by using recording light in a wavelength region shorter than 700 nm and endowed with sufficient recording/readout properties, and a two-photon absorption compound usable therefor.

As a result of intensive studies, the present inventors have found that the above-described object can be attained by the following constructions.
[1] A non-resonant two-photon absorption recording material comprising:

(a) a non-resonant two-photon absorption compound; and

(b) a recording component capable of changing at least either one of a refractive index and a fluorescent intensity,

wherein

the non-resonant two-photon absorption compound (a) is represented by the following formula (1):

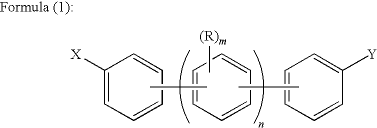

wherein

both of X and Y represent a substituent having a Hammett sigma para value (σp value) of 0 or more and they may be the same as or different from each other;

n represents an integer of 1 to 4;

R represents a substituent and each R may be the same as or different from every other R; and

m represents an integer of 0 to 4.
[2] The non-resonant two-photon absorption recording material of [1] above, wherein

the non-resonant two-photon absorption compound (a) is represented by the following formula (2):

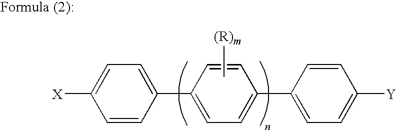

wherein

both of X and Y represent a substituent having a Hammett sigma para value (σp value) of 0 or more and they may be the same as or different from each other;

n represents an integer of 1 to 4;

R represents a substituent and each R may be the same as or different from every other R; and

m represents an integer of 0 to 4.

[3] The non-resonant two-photon absorption recording material of [1] or [29 above, wherein

the non-resonant two-photon absorption compound (a) is represented by the following formula (3):

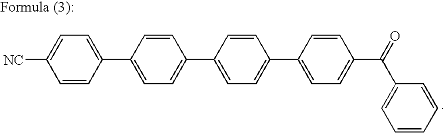

[4] The non-resonant two-photon absorption recording material of [1] or [29 above, wherein

the non-resonant two-photon absorption compound (a) is represented by the following formula (4):

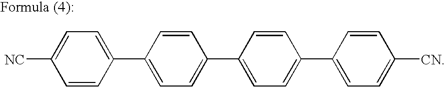

[5] A compound represented by the following formula (3):

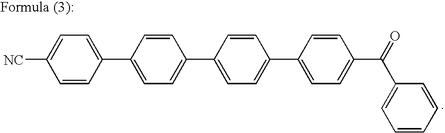

### BRIEF DESCRIPTION OF THE DRAWING

FIG. 1 illustrates a block diagram schematically showing the system for two-photon recording/readout evaluation of a two-photon absorption recording material by the fluorescent intensity change in Example 2;

FIG. 2 illustrates an image of fluorescence signals obtained from recording pits of Two-Photon Absorption Recording Medium 5 in Example 2;

FIG. 3 illustrates an image of fluorescence signals obtained from recording pits of Two-Photon Absorption Recording Medium 6 in Example 2; and

FIG. 4 illustrates a graph showing the relationship between recording light irradiation intensity and fluorescence signal readout intensity in the two-photon recording sensitivity evaluation with recording light of 405 nm of Two-Photon Absorption Recording Medium 5 in Example 2.

### DETAILED DESCRIPTION OF THE INVENTION

The two-photon absorption recording material of the present invention is described in detail below.

### <Non-Resonant Two-Photon Absorption Compound>

The non-resonant two-photon absorption compound (a) for use in the non-resonant two-photon absorption recording material of the present invention is described below.

The non-resonant two-photon absorption compound (a) for use in the non-resonant two-photon absorption recording material of the present invention is a compound having a structure represented by the following formula (1):

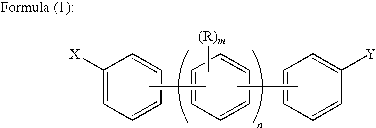

(wherein both of X and Y represent a substituent having a Hammett sigma para value (σp value) of 0 or more and they may be the same as or different from each other; n represents an integer of 1 to 4; R represents a substituent and each R may be the same as or different from every other R; and m represents an integer of 0 to 4).

In formula (1), X and Y each represents a so-called electron-withdrawing group with its Hammett σp value taking a positive value and is preferably, for example, a trifluoromethyl group, a heterocyclic group, a halogen atom, a cyano group, a nitro group, an alkylsulfonyl group, an arylsulfonyl group, a sulfamoyl group, a carbamoyl group, an acyl group, an acyloxy group or an alkoxy carbonyl group, more preferably a trifluoromethyl group, a cyano group, an acyl group, an acyloxy group or an alkoxy carbonyl group, and most preferably a cyano group or a benzoyl group. Out of these substituents, the alkylsulfonyl group, arylsulfonyl group, sulfamoyl group, carbamoyl group, acyl group, acyloxy group and alkoxycarbonyl group each may further have a substituent for imparting solubility in a solvent or other various purposes, and preferred examples of the substituent include an alkyl group, an alkoxy group, an alkoxyalkyl group and an aryloxy group.

n represents an integer of 1 or 4 and is preferably 2 or 3, most preferably 2. As n becomes 5 or more, the linear absorption comes to appear on the long wavelength side, and non-resonant two-photon absorption recording using recording light in a wavelength region shorter than 700 nm cannot be performed.

R represents a substituent, and the substituent is not particularly limited. Specific examples thereof include an alkyl group, an alkoxy group, an alkoxyalkyl group and an aryloxy group. m represents an integer of 0 to 4.

In the compound having a structure represented by formula (1), X and Y are preferably a so-called electron-withdrawing group with its Hammett σp value taking a positive value, and this is described below.

According to T. Kogej, et al., Chem. Phys. Lett., 298, 1 (1998), the two-photon absorption efficiency, that is, the two-photon absorption cross-sectional area δ of an organic compound has the following relationship with the imaginary part of the third-order molecular polarizability (second-order hyperpolarizability) γ.

$$δ ( ω ) = ( \frac{3 π {hv}^{2}}{{n}^{2} {c}^{2} {ɛ}_{0}} ) Im γ ( - ω ; ω , - ω , ω ) \qquad Mathematical Formula ( 1 )$$

wherein c: light speed, ν: frequency, n: refractive index, ε₀: dielectric constant in vacuum, ω: vibration frequency, and Im: imaginary part. The imaginary part (Imγ) of γ has the following relationship with Mge: dipole moment between |g> and |e>, Mge′: dipole moment between |g> and |e′>, Δμge: different of dipole moment between |g> and |e>, Ege: transition energy, and Γ: damping factor.

$$Im γ ( - ω ; ω , - ω , ω ) = Im P [ \begin{matrix}\begin{matrix}\frac{{Mge}^{2} Δμ {ge}^{2}}{\begin{matrix}\begin{matrix}( Ege - ℏω - iΓ ge ) \\ ( Ege - 2 ℏω - iΓ ge )\end{matrix} \\ ( Ege - ℏω - iΓ ge )\end{matrix}} + \\ {\sum }_{{e}^{′}} \frac{{Mge}^{2} {Mee}^{′2}}{\begin{matrix}\begin{matrix}( Ege - ℏω - iΓ ge ) \\ ( {Ege}^{′} - 2 ℏω - iΓ {ge}^{′} )\end{matrix} \\ ( Ege - ℏω - iΓ ge )\end{matrix}} -\end{matrix} \\ \frac{{Mge}^{4}}{\begin{matrix}\begin{matrix}( Ege - ℏω - iΓ ge ) \\ ( Ege + ℏω + iΓ ge )\end{matrix} \\ ( Ege - ℏω - iΓ ge )\end{matrix}}\end{matrix} ] \qquad Mathematical Formula ( 2 )$$

wherein P represents a variable operator.

Accordingly, when the value of mathematical formula (2) is computed, the two-photon absorption cross-sectional area of a compound can be predicted. Consequently, the most stable structure in the ground state is computed by a DFT method using a B3LYP functional with a 6-31G* basis function, and Mge, Mee′ and Ege are computed based on the result, whereby the value of Imγ can be computed. For example, assuming that the maximum Imγ value obtained by the computation of a quaterphenyl compound that is compound having a structure represented by formula (1) where a methoxy group as an electron-donating substituent is substituted on X and Y is 1, the relative value of the maximum Imγ value of a molecule having, as other substituents, a so-called electron-withdrawing group with its Hammett σp value taking a positive value becomes large.

As regards the compound having a structure represented by formula (1), Imγ is small in the case of a quaterphenyl compound where a methoxy group as an electron-donating group is substituted on X and Y, and Imγ greatly increases in general in the case of a molecule where an electron-withdrawing substituent is substituted on both X and Y. As described above, the two-photon absorption cross-sectional area δ is theoretically proportional to the imaginary part of the third-order hyperpolarizability γ, that is, Imγ, and judging from the computation thereof, a structure where an electron-withdrawing substituent is substituted on both X and Y is preferred.

The compound having a structure represented by formula (1) is preferably a compound having a structure represented by the following formula (2):

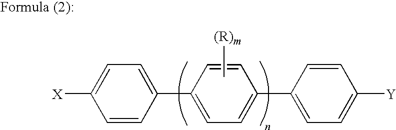

In formula (2), X, Y, n, R and m are the same as those specified in formula (1).

In the compound having a structure represented by formula (1) or (2), X and Y may be the same as or different from each other, but are preferably different, because a large two-photon absorption cross-sectional area tend to be obtained.

Specific examples of the compound having a structure represented by formula (1) or (2) are not particularly limited but include those set forth below.

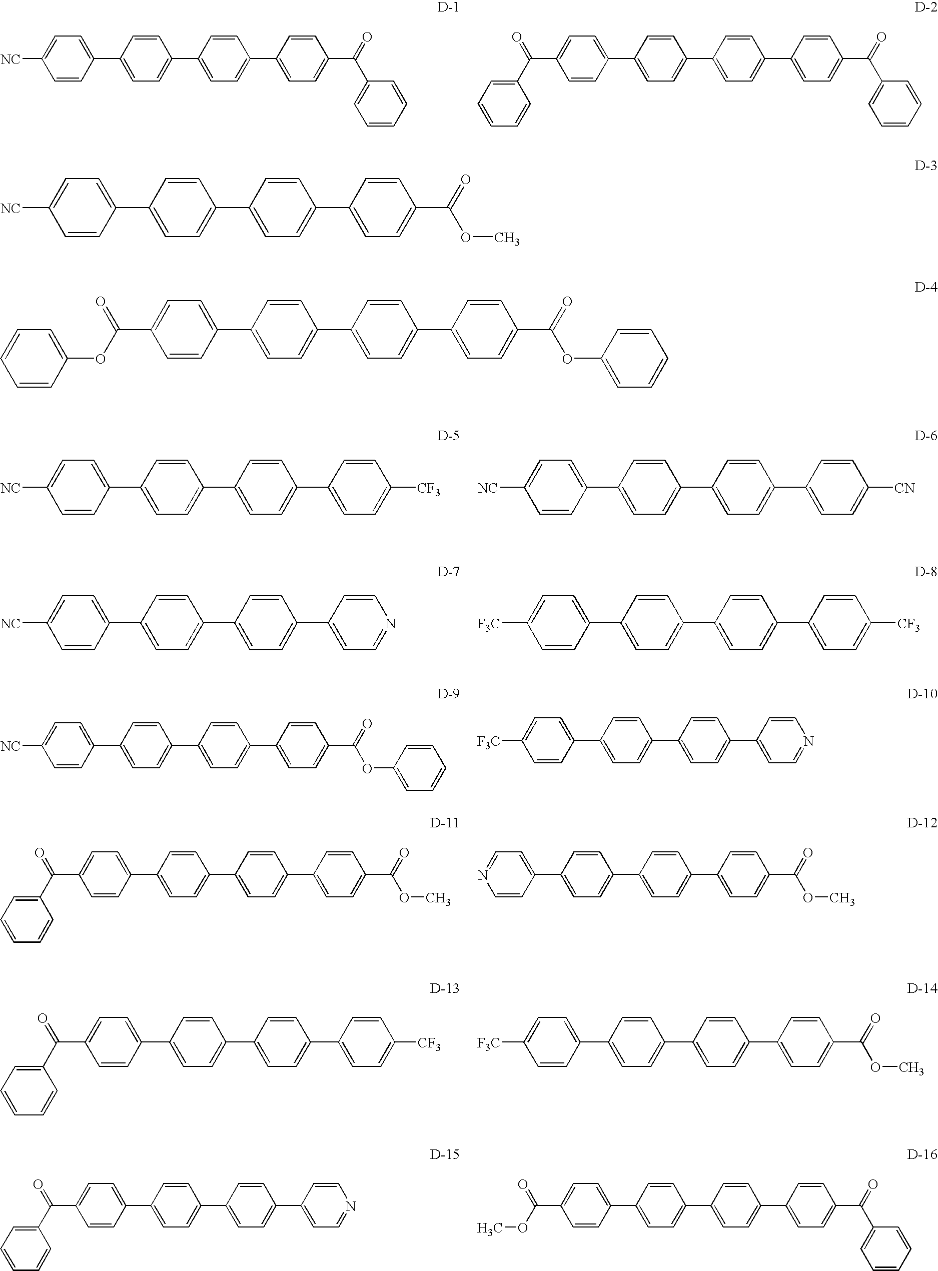

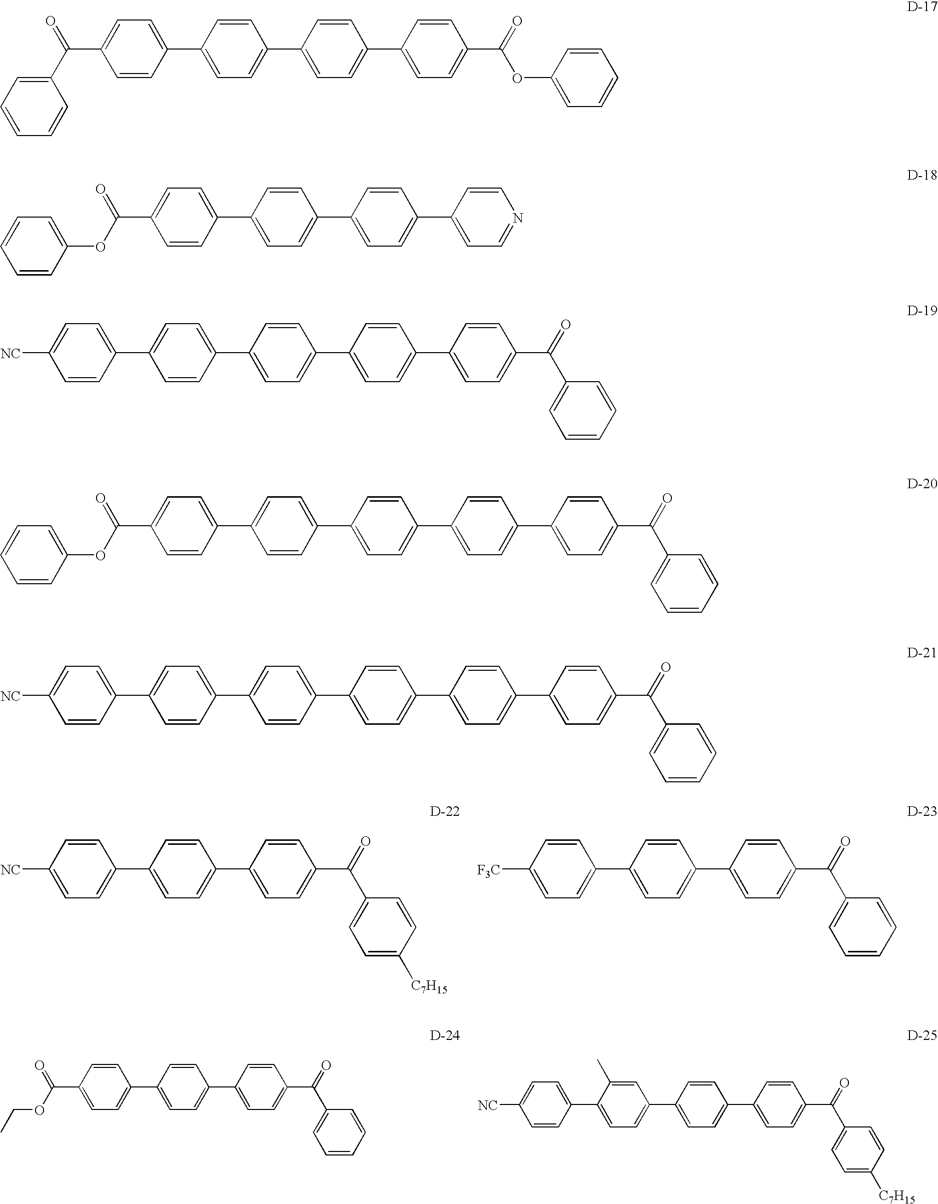

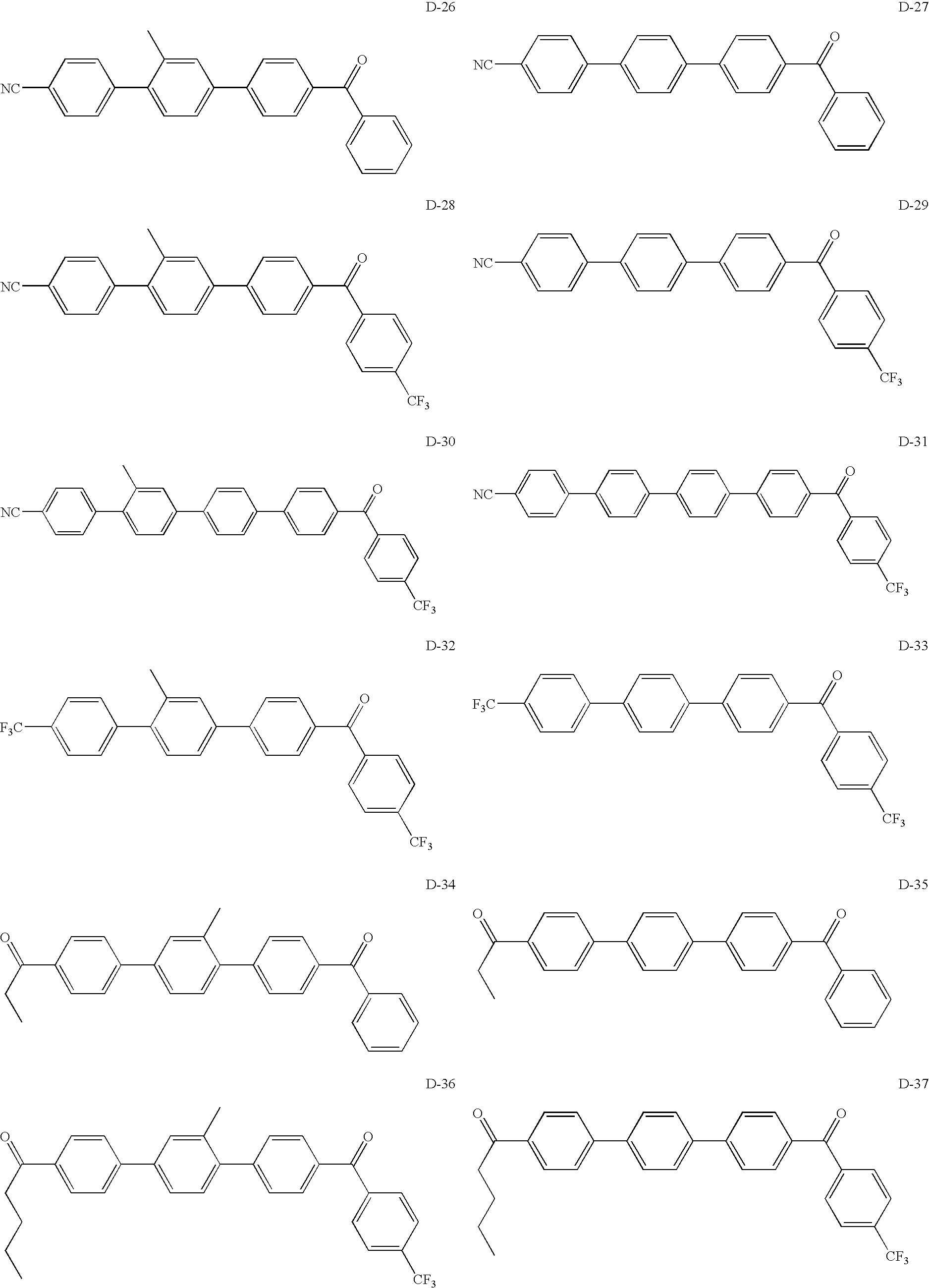

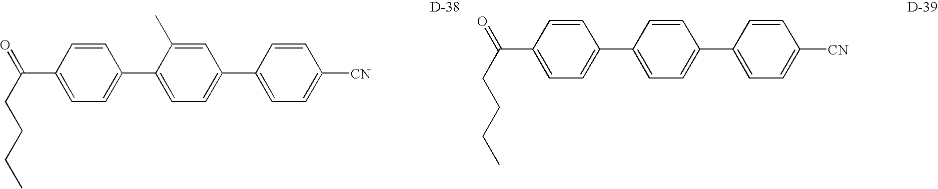

Among the compounds above, Compound D-1 is preferred. Compound D-1 is a novel compound.

### <Recording Component Capable of Changing at Least Either One of a Refractive Index and a Fluorescent Intensity>

As the recording component (b) capable of changing at least either one of a refractive index and a fluorescent intensity, for use in the non-resonant two-photon absorption recording material of the present invention, exemplified are:

(I) a material that creates a refractive index or fluorescence modulation through color formation of a dye or a fluorescent dye;

(II) a material that creates a refractive index modulation through polymerization;

(III) a material that creates a refractive index modulation through polymerization of a dye having a polymerizable group;

(IV) a material that forms a latent image capable of creating a refractive index/fluorescence modulation through color formation of a dye; and

(V) a material that forms a latent image capable of creating a refractive index/fluorescence modulation through polymerization.

These materials are described below

[Material that Creates a Refractive Index or Fluorescence Modulation Through Color Formation of a Dye or a Fluorescent Dye]

The material that creates a refractive index or fluorescence modulation through color formation of a dye or a fluorescent dye preferably contains, for example, at least one or more members out of:

(A) a dye precursor whose absorption band comes to appear in the visible region by the action of an acid,

(B) a dye precursor whose absorption band comes to appear in the visible region by the action of a base,

(C) a dye precursor whose absorption band appears in the visible region resulting from oxidation,

(D) a dye precursor whose absorption appears in the visible region resulting from reduction.

These dye precursors are described below.

### (A) Dye Precursor Whose Absorption Band Comes to Appear in the Cisible Region by the Action of an Acid

This dye precursor is a dye precursor capable of becoming a color former whose absorption is changed from the original state, by the action of an acid generated from an acid generator. The acid-color forming precursor is preferably a compound whose absorption is shifted to the long wavelength side by the action of an acid, more preferably a compound which is colorless but color-formed by the action of an acid.

Preferred examples of the acid-color forming dye precursor include a triphenylmethane-based compound, a phthalide-based compound (including indolylphthalide-based, azaphthalide-based and triphenylmethane phthalide-based compounds), a phenothiazine-based compound, a phenoxazine-based compound, a fluorane-based compound, a thiofluorane-based compound, a xanthene-based compound, a diphenylmethane-based compound, a chromenopyrazole-based compound, leucoauramine, a methine-based compound, an azomethine-based compound, a Rhodamine lactam-based compound, a quinazoline-based compound, a diazaxanthene-based compound, a fluorene-based compound and a spiropyran-based compound. Specific examples of these compounds are disclosed, for example, in JP-A-2002-156454, patents cited therein, JP-A-2000-281920, JP-A-11-279328 and JP-A-8-240908.

The acid-color forming dye precursor is more preferably a leuco dye having a partial structure such as lactone, lactam, oxazine or spiropyran, and examples thereof include fluorane-based, thiofluorane-based, phthalide-based, Rhodamine lactam-based and spiropyran-based compounds. The acid-color forming dye precursor is still more preferably a xanethene (fluorane) dye or a triphenylmethane dye. If desired, two or more of these acid-color forming dye precursors may be used as a mixture in an arbitrary ratio.

As for specific preferred examples of the acid-color forming dye precursor, there may be used compounds disclosed in JP-A-2007-87532, that is, compounds represented by formulae (21) to (23) and described in paragraph 0122 (phthalide-based dye precursors (including indolylphthalide-based dye precursors and azaphthalide-based dye precursors)), compounds represented by formula (24) and described in paragraph 0126 (triphenylmethane phthalide-based dye precursors), compounds represented by formula (25) and described in paragraph 0130 (fluorane-based dye precursors), compounds described in paragraph 0131 (Rhodamine lactam-base dyed precursors), and compounds described in paragraph 0132 (spiropyran-based dye precursors).

Furthermore, as for the acid-color forming dye precursor, BLD compounds represented by formula (6) of JP-A-2008-284475, leuco dyes disclosed in JP-A-2000-144004, and leuco dyes having structures set forth in [Chem. 38] of JP-A-2007-87532 may also be suitably used.

In addition, compounds represented by formula (26) and set forth in [Chem. 40] of JP-A-2007-87532, which can form a color by the addition of an acid (proton), may also be used as the dye precursor.

Specific preferred examples of the acid-color forming dye precursor include those compounds described in JP-A-2007-87532, supra, but the present invention is not limited thereto.

### (B) Dye Precursor Whose Absorption Band Comes to Appear in the Visible Region by the Action of a Base

This dye precursor is a dye precursor capable of becoming a color former whose absorption is changed from the original state, by the action of a base generated from a base generator.

The base-color forming dye precursor for use in the present invention is preferably a compound whose absorption is shifted to the long wavelength side by the action of a base, more preferably a compound whose molar extinction coefficient is greatly increased by the action of a base.

The base-color forming dye precursor for use in the present invention is preferably a dissociative dye in the non-dissociated form. The dissociative dye is a compound where a dissociative group having a pKa of 12 or less, preferably 10 or less, and readily dissociating to release a proton is present on a dye chromophore and when changed from a non-dissociated form to a dissociated form, the absorption is shifted to the long wavelength side or the colorless state turns to the color-formed state. Preferred examples of the dissociative group include an OH group, an SH group, a COOH group, a PO₃H₂ group, an SO₃H group, an NR⁹¹R⁹²H⁺ group, an NHSO₂R⁹³ group, a CHR⁹⁴R⁹⁵ group and an NHR⁹⁶ group.

Here, each of R⁹¹, R⁹² and R⁹⁶ independently represents a hydrogen atom, an alkyl group (preferably having a C number of 1 to 20, e.g., methyl, ethyl, n-propyl, isopropyl, n-butyl, n-pentyl, benzyl, 3-sulfopropyl, 4-sulfobutyl, carboxymethyl, 5-carboxypentyl), an alkenyl group (preferably having a C number of 2 to 20, e.g., vinyl, allyl, 2-butenyl, 1,3-butadienyl), a cycloalkyl group (preferably having a C number of 3 to 20, e.g., cyclopentyl, cyclohexyl), an aryl group (preferably having a C number of 6 to 20, e.g., phenyl, 2-chlorophenyl, 4-methoxyphenyl, 3-methylphenyl, 1-naphthyl), or a heterocyclic group (preferably having a C number of 1 to 20, e.g., pyridyl, thienyl, furyl, thiazolyl, imidazolyl, pyrazolyl, pyrrolidino, piperidino, morpholino), preferably a hydrogen atom or an alkyl group. R⁹³ represents an alkyl group, an alkenyl group, a cycloalkyl group, an aryl group or a heterocyclic group (preferred examples of the substituent are the same as those of the substituent for R⁹¹, R⁹² and R⁹⁶), preferably an alkyl group which may be substituted, or an aryl group which may be substituted, more preferably an alkyl group which may be substituted, and the substituent here preferably has electron-withdrawing property and is preferably fluorine.

Each of R⁹⁴ and R⁹⁵ independently represents a substituent (preferred examples of the substituent are the same as those of the substituent for R⁹¹, R⁹² and R⁹⁶), but an electron-withdrawing substituent is preferred and this is preferably a cyano group, an alkoxycarbonyl group, a carbamoyl group, an acyl group, an alkylsulfonyl group or an arylsulfonyl group.

The dissociative group in the dissociative dye for use in the present invention is preferably an OH group, an SH group, a COOH group, a PO₃H₂ group, an SO₃H group, an NR⁹¹R⁹²H⁺ group, an NHSO₂R⁹³ group or a CHR⁹⁴R⁹⁵ group, more preferably an OH group or a CHR⁹⁴R⁹⁵ group, and most preferably an OH group.

The dissociative dye in the non-dissociated form as the base-color forming dye precursor for use in the present invention is preferably a non-dissociated form of dissociative azo dye, dissociative azomethine dye, dissociative oxonol dye, dissociative arylidene dye, dissociative xanthene (fluorane) dye or dissociative triphenylamine dye, more preferably a non-dissociated form of dissociative azo dye, dissociative azomethine dye, dissociative oxonol dye or dissociative arylidene dye.

Specific preferred examples of the base-color forming dye precursor include the compounds described in JP-A-2007-87532, paragraphs 0144 to 0146, but the present invention is not limited thereto.

(C) Dye Precursor Whose Absorption Band Appears in the Visible Region Resulting from Oxidation

This dye precursor is not particularly limited as long as it is a compound whose absorbance is increased by an oxidation reaction, but the dye precursor preferably contains at least one or more kinds of compounds selected from leuco quinone compounds, thiazine leuco compounds, oxazine leuco compounds, phenazine leuco compounds and leuco triarylmethane compounds.

As for the leuco quinone compounds, compounds having a partial structure represented by formulae (6) to (10) and set forth in paragraphs 0149 and 0150 of JP-A-2007-87532 may be used.

As for the thiazine leuco compounds, oxazine leuco compounds and phenoxazine leuco compounds, compounds represented by formulae (11) and (12) and set forth in paragraphs 0156 to 0160 of JP-A-2007-87532 may be used.

As for the leuco triarylmethane compounds, compounds having a partial structure represented by formula (13) and set forth in paragraphs 0166 and 0167 of JP-A-2007-87532 are preferred.

Specific preferred examples of the dye precursor whose absorption band appears in the visible region resulting from oxidation, which is used in the present invention, include compounds set forth in paragraph 0152 (leuco quinone compounds), paragraphs 0162 to 0164 (thiazine leuco compounds, oxazine leuco compounds and phenazine leuco compounds) and paragraphs 0169 and 0170 (leuco triarylmethane compounds) of JP-A-2007-87532, but the present invention is not limited thereto.

(D) Dye Precursor Whose Absorption Appears in the Visible Region Resulting from Reduction

As for this dye precursor, compounds represented by formula (A) of JP-A-2007-87532 may be used, and specifically, compounds set forth in paragraphs 0172 to 0195 of the same patent publication may be used.

Here, when the recording component for use in the present invention contains the above-described dye precursor, it is also preferred that the two-photon absorption optical recording material of the present invention further contains a base, if desired, for the purpose of dissociating the produced dissociative dye. The base may be either an organic base or an inorganic base, and preferred examples thereof include alkylamines, anilines, imidazoles, pyridines, carbonates, hydroxide salts, carboxylates and metal alkoxide. Also, polymers containing such a base are preferably used.

Incidentally, the dye precursor for use in the present invention may be a commercially available product or may be synthesized by a known method.

In the two-photon recording process, the spectral change due to color formation of a dye precursor in the site subjected to recording by two-photon absorption recording preferably appears in the wavelength region longer than the maximum wavelength in the linear absorption spectrum of the two-photon absorption dye. Alternatively, it is preferred that the absorption spectral change appears in the wavelength region shorter than the readout wavelength and at the same time, the absorption spectral change is not present at the readout wavelength. Thanks to such a construction, a recording signal can be efficiently read out from the reflected light by using a large change of the refractive index, which is attributable to abnormal refractive index dispersion appearing as a result of color formation of a dye and appears on the longer wavelength side than the absorption maximum wavelength of the color-forming dye.

In the two-photon recording process, it is preferred that the spectral change due to decoloration of a dye in the site subjected to recording by two-photon absorption recording appears at the readout wavelength or in the wavelength region shorter than the readout wavelength and the dye absorption is not present at the readout wavelength. Thanks to such a construction, the refractive index change at the readout wavelength can be increased and a recording signal can be efficiently read out from the reflected light.

The optical recording material of the present invention can contain, as the component other than the above-described components, an electron-donating compound capable of donating an electron to the two-photon absorption compound and/or a compound constituting the recording component, an acid generator and a base generator, if desired. Compounds described in paragraphs 0199 to 0217 of JP-A-2007-87532 may be used as the electron-donating compound; compounds described in paragraphs of 0218 to 0245 of the same patent publication may be used as the acid generator; and compounds described in paragraphs 0246 to 0267 of the same patent publication may be used as the base generator.

The material that creates a refractive index or fluorescence modulation through color formation of a dye or a fluorescent dye is described in more detail in JP-A-2007-87532.

[Material that Creates a Refractive Index Modulation Through Polymerization]

The material that creates a refractive index modulation through polymerization is composed of at least a polymerizable compound and a polymerization initiator. The material is described in detail below.

### (Polymerizable Compound)

The polymerizable compound is a compound capable of causing addition polymerization by the action of a radical or an acid (Broensted acid or Lewis acid) and thereby undertaking oligomerization or polymerization.

The polymerizable compound may be monofunctional or polyfunctional, may comprise one component or multiple components, or may be a monomer, a prepolymer (e.g., dimer, oligomer) or a mixture thereof. Also, its form may be a liquid or a solid.

The polymerizable compounds are roughly classified into a polymerizable compound capable of radical polymerization and a polymerizable compound capable of cationic polymerization.

The radical polymerizable compound is preferably a compound having at least one ethylenically unsaturated double bone within the molecule, and specific examples thereof include the polymerizable monomers described below and prepolymers (e.g., dimer, oligomer) comprising such a polymerizable monomer. These may be monofunctional type or polyfunctional type. Examples thereof include an ethylenically unsaturated acid compound, an aliphatic or aromatic functional group-containing (meth)acrylate, and an amide monomer of an unsaturated carboxylic acid with an aliphatic polyvalent amine compound. As for specific examples, compounds described in paragraphs 0019 to 0026 of JP-A-2005-29725 may be used.

Furthermore, as for the radical polymerizable compound, compounds described in paragraph 0027 (polyisocyanate compounds), paragraph 0028 (urethane acrylates), paragraph 0030 (phosphorus-containing monomers) and paragraphs 0031 to 0032 (commercial products) of JP-A-2005-29725 may be used. In addition, those described as a photocurable monomer or an oligomer in Journal of the Adhesion Society of Japan, Vol. 20, No. 7, pp. 300-30 may also be used.

The cationic polymerizable compound is a compound of starting its polymerization under the action of an acid generated by the two-photon absorption compound and the cationic polymerization initiator, and examples thereof include compounds described in J. V. Crivello, Chemtech. Oct., page 624 (1980), JP-A-62-149784, and Journal of the Adhesion Society of Japan, Vol. 26, No. 5, pp. 179-187 (1990).

The cationic polymerizable compound is preferably a compound having at least one oxirane ring, oxetane ring or vinyl ether group moiety within the molecule, more preferably a compound having an oxirane ring. Specifically, the cationic polymerizable compound includes the following cationic polymerizable monomers and prepolymers (e.g., dimer, oligomer) comprising such a cationic polymerizable monomer.

Specific examples of the cationic polymerizable monomer having an oxirane ring include compounds described in paragraphs 0035 to 0036 of JP-A-2005-29725.

Specific examples of the cationic polymerizable monomer having an oxetane ring include compounds described above as specific examples of the cationic polymerizable monomers having an oxirane ring, where the oxirane ring is replaced by an oxetane ring. Specifically, the monomer includes compounds described in paragraph 0038 of JP-A-2005-29725.

### (Polymerization Initiator)

The polymerization initiator is described below. The polymerization initiator for use in the present invention is a compound capable of generating a radical or an acid (Broensted acid or Lewis acid) as a result of energy or electron transfer (giving or accepting an electron) from the excited state of the two-photon absorption compound, which is produced upon two-photon absorption, and thereby initiating polymerization of the polymerizable compound.

The polymerization initiator for use in the present invention is preferably a radical polymerization initiator capable of generating a radical and initiating radical polymerization of the polymerizable compound, a cationic polymerization initiator capable of generating only an acid without generating a radical and initiating only cationic polymerization of the polymerizable compound, or a polymerization initiator capable of generating both a radical and an acid and initiating both radical polymerization and cationic polymerization.

As for the polymerization initiator, the following 14 systems are preferred. These polymerization initiators may be used, if desired, as a mixture of two or more thereof in an arbitrary ratio.
1) Ketone-based polymerization initiator
2) Organic peroxide-based polymerization initiator
3) Bisimidazole-based polymerization initiator
4) Trihalomethyl-substituted triazine-based polymerization initiator
5) Diazonium salt-based polymerization initiator
6) Diaryliodonium salt-based polymerization initiator
7) Sulfonium salt-based polymerization initiator
8) Borate-based polymerization initiator
9) Diaryliodonium organic boron complex-based polymerization initiator
10) Sulfonium organic boron complex-based polymerization initiator
11) Metal arene complex-based polymerization initiator
12) Sulfonic acid ester-based polymerization initiator

Preferred examples of the polymerization initiator include compounds described in paragraphs 0117 to 0120 (ketone-based polymerization initiators), paragraph 0122 (organic peroxide-based polymerization initiators), paragraphs 0124 and 0125 (bisimidazole-based polymerization initiators), paragraphs 0127 to 0130 (trihalomethyl-substituted triazine-based polymerization initiators), paragraphs 0132 to 0135 (diazonium salt-based polymerization initiators), paragraphs 0137 to 0140 (diaryliodonium salt-based polymerization initiators), paragraphs 0142 to 0145 (sulfonium salt-based polymerization initiators), paragraphs 0147 to 0150 (borate-based polymerization initiators), paragraphs 0153 to 0157 (diaryliodonium organic boron complex-based polymerization initiators), paragraphs 0159 to 0164 (sulfonium organic boron complex-based polymerization initiators), paragraph 0179 (metal arene-based polymerization initiators) and paragraphs 0181 and 0182 (sulfonic acid ester-based polymerization initiators) of JP-A-2005-29725.
13) Other polymerization initiators

Examples of the polymerization initiator other than 1) to 12) above include an organic azide compound such as 4,4′-diazide chalcone, an aromatic carboxylic acid such as N-phenylglycine, a polyhalogen compound (e.g., CI₄, CHI_{S}, CBrCI₃), phenylisoxazolone, a silanol aluminum complex, and aluminate complexes described in JP-A-3-209477.

Here, the polymerization initiators for use in the present invention can be classified into:

a) a polymerization initiator capable of activating radical polymerization,

b) a polymerization initiator capable of activating only cationic polymerization, and

c) a polymerization initiator capable of simultaneously activating radical polymerization and cationic polymerization.

The polymerization initiator a) capable of activating radical polymerization is a polymerization initiator capable of generating a radical as a result of energy or electron transfer (giving an electron to the two-photon absorption compound or accepting an electron from the two-photon absorption compound) from the excited state of the two-photon absorption compound, which is produced upon non-resonant two-photon absorption, and thereby initiating radical polymerization of the polymerizable compound.

Out of the above-described systems, the following systems come under the polymerization initiator capable of activating radical polymerization: 1) ketone-based polymerization initiator, 2) organic peroxide-based polymerization initiator, 3) bisimidazole-based polymerization initiator, 4) trihalomethyl-substituted triazine-based polymerization initiator, 5) diazonium salt-based polymerization initiator, 6) diaryliodonium salt-based polymerization initiator, 7) sulfonium salt-based polymerization initiator, 8) borate-based polymerization initiator, 9) diaryliodonium organic boron complex-based polymerization initiator, 10) sulfonium organic boron complex-based polymerization initiator, and 11) metal arene complex-based polymerization initiator.

Among these polymerization initiators capable of activating radical polymerization, preferred are 1) ketone-based polymerization initiator, 3) bisimidazole-based polymerization initiator, 4) trihalomethyl-substituted triazine-based polymerization initiator, 6) diaryliodonium salt-based polymerization initiator, and 7) sulfonium salt-based polymerization initiator, and more preferred are 3) bisimidazole-based polymerization initiator, 6) diaryliodonium salt-based polymerization initiator, and 7) sulfonium salt-based polymerization initiator.

The polymerization initiator capable of activating only cationic polymerization is a polymerization initiator capable of generating an acid (Broensted acid or Lewis acid) without generating a radical as a result of energy or electron transfer from the excited state of the two-photon absorption compound, which is produced upon non-resonant two-photon absorption, and initiating cationic polymerization of the polymerizable compound by the action of the acid.

Out of the above-described systems, the following system comes under the polymerization initiator capable of activating only cationic polymerization: 14) sulfonic acid ester-based polymerization initiator.

Here, as regards the cationic polymerization initiator, those described, for example, in S. Peter Pappas (compiler), UV Curing; Science and Technology, pp. 23-76, A Technology Marketing Publication; and B. Klingert, M. Riediker and A. Roloff, Comments Inorg. Chem., Vol. 7, No. 3, pp. 109-138 (1988) can also be used.

The polymerization initiator capable of simultaneously activating radical polymerization and cationic polymerization is a polymerization initiator capable of simultaneously generating a radical and an acid (Broensted acid or Lewis acid) as a result of energy or electron transfer from the excited state of the two-photon absorption compound, which is produced upon non-resonant two-photon absorption, and initiating radical polymerization of the polymerizable compound by the action of the radical generated and also cationic polymerization of the polymerizable compound by the action of the acid generated.

Out of the above-described systems, the following systems come under the polymerization initiator capable of simultaneously activating radical polymerization and cationic polymerization: 4) trihalomethyl-substituted triazine-based polymerization initiator, 5) diazonium salt-based polymerization initiator, 6) diaryliodonium salt-based polymerization initiator, 7) sulfonium salt-based polymerization initiator, and 13) metal arene complex-based polymerization initiator.

Among these polymerization initiators capable of activating radical polymerization and cationic polymerization, preferred are 6) diaryliodonium salt-based polymerization initiator and 7) sulfonium salt-based polymerization initiator.

The material that creates a refractive index modulation through polymerization is described in more detail in JP-A-2005-29725.

[Material that Creates a Refractive Index Modulation Through Polymerization of a Dye having a Polymerizable Group]

Also, a material that creates a refractive index modulation through polymerization of a dye having a polymerizable group (sometimes referred to as a “dye monomer”) can be used.

### (Dye Monomer)

The “dye” in the dye monomer indicates a compound that absorbs ultraviolet light, visible light or infrared light at a wavelength of 300 to 2,000 nm, preferably a compound that absorbs ultraviolet light or visible light at a wavelength of 330 to 700 nm, more preferably a compound that absorbs visible light at a wavelength of 400 to 700 nm. The molar extinction coefficient in this region is preferably 5,000 or more, more preferably 10,000 or more, and most preferably 20,000 or more.

In the case of using a dye monomer, the material contains at least a sensitizing dye, a polymerization initiator and a binder, in addition to the dye monomer, and preferably further contains a polymerizable compound not having a dye moiety. Examples of the polymerization initiator and the polymerizable compound not having a dye moiety are the same as those described above.

In the case of using a dye monomer, the recording method is characterized in that polymerization of the dye having a polymerizable group and the polymerizable compound not having a dye moiety is brought about by activating the polymerization initiator through electron or energy transfer from the excited state of the two-photon absorption compound, which is produced resulting from absorption of light upon irradiation of two-photon recording light, and a refractive index modulation is created on this occasion by allowing mainly the dye having a polymerizable group and the polymerizable compound not having a dye moiety to move in the light-irradiated part while expelling mainly the binder to the light-unirradiated part, whereby a recording pit is recorded.

Accordingly, in this case, the refractive index of the dye having a polymerizable group at the readout wavelength is preferably larger than that of the binder.

In general, the refractive index of a dye takes a high value in the region from the neighborhood of absorption maximum wavelength (λmax) to a wavelength longer than that and takes a very high value particularly in the region from λmax to a wavelength about 200 nm longer than λmax, and depending on the dye, the refractive index takes as a high value as exceeding 2, even exceeding 2.5.

On the other hand, the refractive index of an organic compound that is not a dye, such as binder polymer, is usually on the order of 1.4 to 1.6.

In this respect, a dye having a large refractive index is used for the refractive index modulation, which is advantageous in view of obtaining high sensitivity. Also, for obtaining high sensitivity, the dye having a polymerizable group preferably gives an absorption spectrum having λmax shorter by 10 to 200 nm, more preferably λmax shorter by 30 to 130 nm, than the hologram readout wavelength and showing that ε is 10,000 or more, more preferably 20,000 or more.

Also in the case of using a polymerizable compound not having a dye moiety, the refractive index of the polymerizable compound not having a dye moiety at a readout wavelength is preferably larger than that of the binder.

In this regard, it is preferred that the polymerizable compound not having a dye moiety contains at least one or more members of an aryl group, an aromatic heterocyclic group, chlorine atom, bromine atom, iodine atom and sulfur atom and the binder does not contain such a group or atom.

As for the polymerization reaction, the polymerization reaction is preferably radical polymerization, cationic polymerization or anionic polymerization, more preferably radial polymerization or cationic polymerization.

At this time, as for the polymerizable group of the dye having a polymerizable group or the polymerizable compound not having a dye moiety, in the case where the polymerization is radical polymerization, the dye or compound preferably contains, as the polymerizable group, an ethylenically unsaturated group moiety such as acryloyl group, methacryloyl group, styryl group or vinyl group, more preferably an acryloyl group or a methacryloyl group, and in the case where the polymerization is cationic polymerization or anionic polymerization, the dye or compound preferably contains, as the polymerizable group, an oxirane ring, an oxetane ring, a vinyl ether group or an N-vinylcarbazole moiety, more preferably an oxirane ring or an oxetane ring.

The dye having a polymerizable group is described in detail below. In the dye having a polymerizable group, preferred examples of the dye moiety include a cyanine dye, a squarylium cyanine dye, a styryl dye, a pyrylium dye, a merocyanine dye, an arylidene dye, an oxonol dye, an azulenium dye, a coumarin dye, a ketocoumarin dye, a styryl coumarin dye, a pyran dye, a xanthene dye, a thioxanthene dye, a phenothiazine dye, a phenoxazine dye, a phenazine dye, a phthalocyanine dye, an azaporphyrin dye, a porphyrin dye, a condensed aromatic dye, a perylene dye, an azomethine dye, an anthraquinone dye, a metal complex dye and an azo dye. Among these dyes, more preferred are a cyanine dye, a squarylium cyanine dye, a styryl dye, a merocyanine dye, an arylidene dye, an oxonol dye, a coumarin dye, a xanthene dye, a phenothiazine dye, a condensed aromatic dye and an azo dye, and still more preferred are a cyanine dye, a merocyanine dye, an arylidene dye, an oxonol dye, a coumarin dye, a xanthene dye and an azo dye.

Other than these, coloring matters and dyes described in Shinya Ohkawara (compiler), Shikiso Handbook (Handbook of Coloring Matters), Kodansha (1986), Shinya Ohkawara (compiler), Kinosei Shikiso no Kagaku (Chemistry of Functional Coloring Matters), CMC (1981), and Chuzaburo Ikemori et al. (compilers), Tokushu Kino Zairyo (special Functional Materials), CMC (1986) can also be used as the dye moiety.

The polymerizable group of the dye having a polymerizable group is as described above, and the polymerizable group may be substituted on any portion of the dye.

Specific examples of the dye having a polymerizable group are set forth below, but the present invention is not limited thereto.

| | |
|---|---|
| DM-1 | 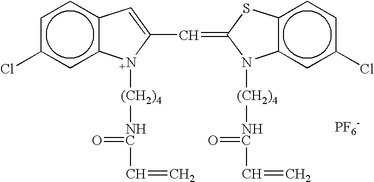 |

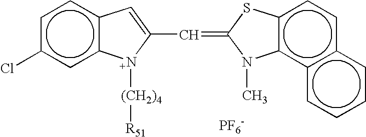

R₅₁

| DM-2 | —NHCOCH═CH₂ |
|---|---|
| DM-3 | 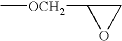 |
| DM-4 | —OCH═CH₂ |
| DM-5 | 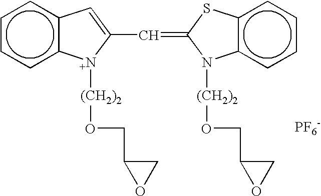 |

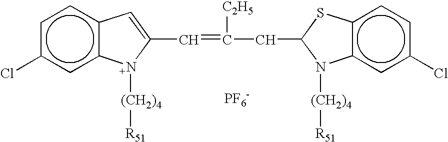

R₅

| DM-6 | 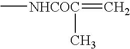 |
|---|---|
| DM-7 | 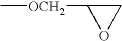 |
| DM-8 | 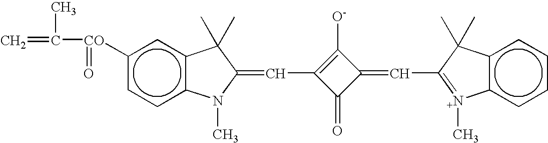 |
| DM-9 | 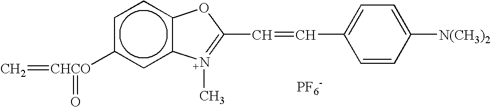 |

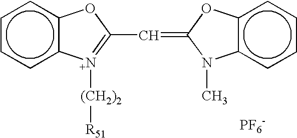

R₅₁

| | |
|---|---|
| DM-10 | 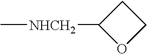 |
| DM-11 | 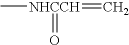 |

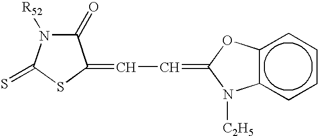

R₅₂

| | |
|---|---|
| DM-12 | —CH═CH₂ |
| DM-13 | 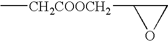 |
| DM-14 | 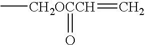 |

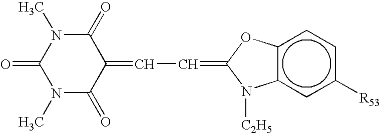

R₅₃

| | |
|---|---|
| DM-15 | 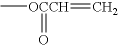 |
| DM-16 | 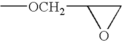 |
| DM-17 | 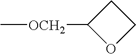 |

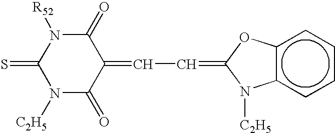

R₅₂

| | |
|---|---|
| DM-18 | —CH═CH₂ |
| DM-19 | 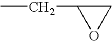 |

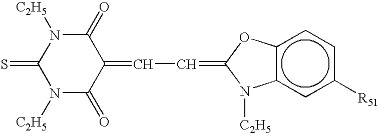

R₅₁

| DM-20 | 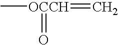 |
|---|---|
| DM-21 | 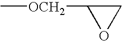 |
| DM-22 | 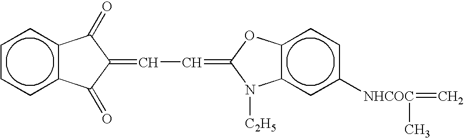 |
| DM-23 | 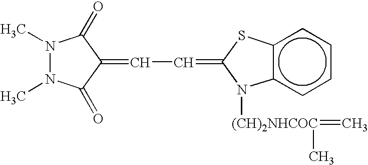 |
| DM-24 | 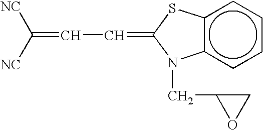 |
| DM-25 | 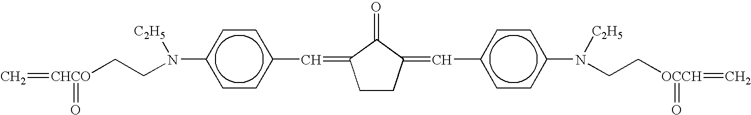 |
| DM-26 | 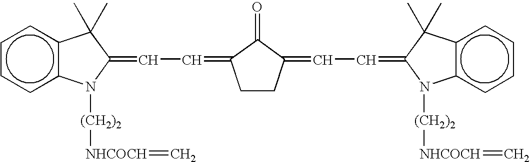 |

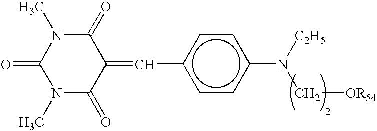

R₅₄

| DM-27 | 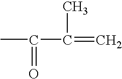 |
|---|---|
| DM-28 | 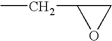 |
| DM-29 | 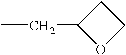 |
| DM-30 | 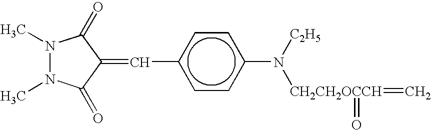 |

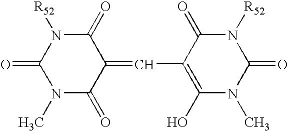

R₅₂

| | |
|---|---|
| DM-31 | —CH═CH₂ |
| DM-32 | 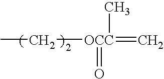 |
| DM-33 | 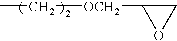 |
| DM-34 | 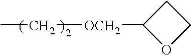 |

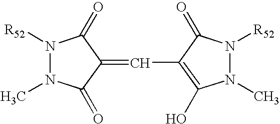

R₅₂

| DM-35 | —CH═CH₂ |
|---|---|
| DM-36 | 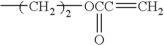 |
| DM-37 | 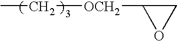 |
| M-1 | 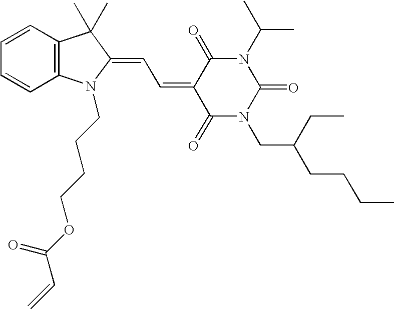 |
| M-2 | 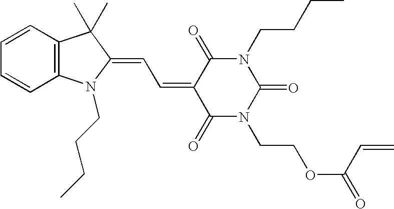 |
| M-3 | 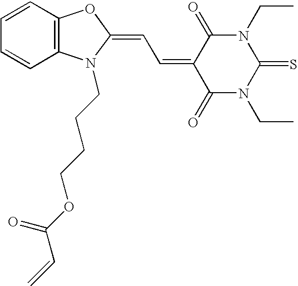 |
| M-4 | 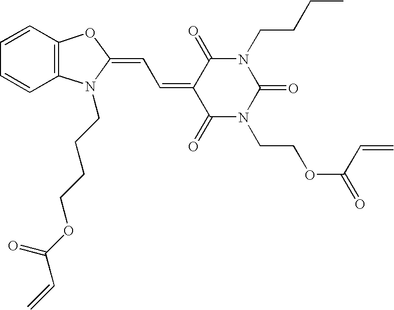 |
| M-5 | 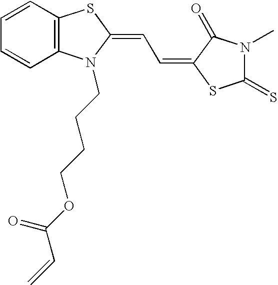 |
| M-6 | 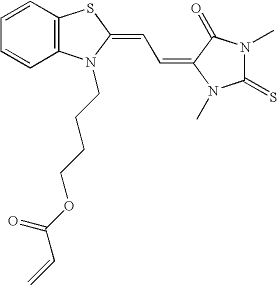 |
| M-7 | 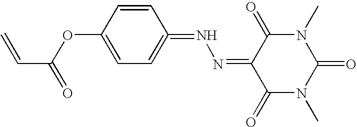 |
| M-8 | 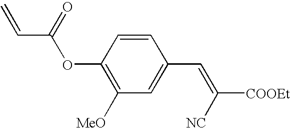 |
| M-9 | 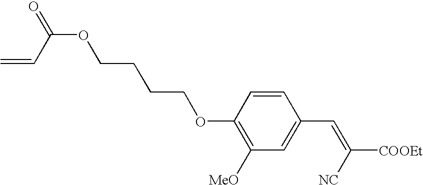 |
| M-10 | 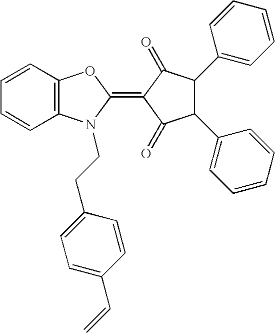 |
| M-11 | 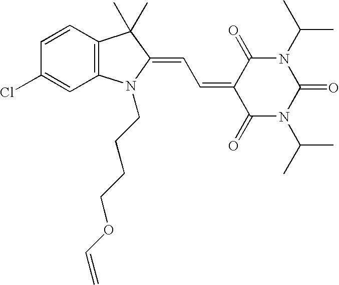 |

### (Binder)

The binder used in combination with the dye monomer is usually used for the purpose of enhancing the film-forming property of the composition before polymerization, the uniformity of film thickness, or the stability during storage. The binder preferably has good compatibility with the polymerizable compound, polymerization initiator and two-photon absorption compound.

The binder is preferably a solvent-soluble thermoplastic polymer, and one of these polymers may be used alone or several kinds thereof may be used in combination.

As described above, the refractive index of the binder used in combination with the dye monomer preferably differs from that of the polymerizable compound, and the polymerizable compound may have a larger refractive index or the binder may have a larger refractive index, but the refractive index of the polymerizable compound is preferably larger than that of the binder.

For this purpose, it is preferred that either one of the polymerizable compound and the binder contains at least one member of an aryl group, an aromatic heterocyclic group, chlorine atom, bromine atom, iodine atom and sulfur atom and the remaining does not contain such a group or atom. More preferably, the polymerizable group contains at least one member of an aryl group, an aromatic heterocyclic group, chlorine atom, bromine atom, iodine atom and sulfur atom and the binder does not contain such a group or atom.

Preferred examples of the binder when the refractive index of the polymerizable compound is larger than the refractive index of the binder are described below.

Specific preferred examples of the low refractive index binder include an acrylate, an α-alkyl acrylate ester, an acidic polymer, an interpolymer (for example, polymethyl methacrylate, polyethyl methacrylate, and a copolymer of methyl methacrylate and another alkyl(meth)acrylate ester), a polyvinyl ester (e.g., polyvinyl acetate, polyvinyl acetate/acrylate, polyvinyl acetate/methacrylate, hydrolyzable polyvinyl acetate), an ethylene/vinyl acetate copolymer, a saturated or unsaturated polyurethane, a butadiene or isoprene polymer or copolymer, a high molecular weight polyethylene oxide of polygycol having an average molecular weight of about 4,000 to 1,000,000, an epoxidized product (for example, an epoxidized product having an acrylate or methacrylate group), a polyamide (e.g., N-methoxymethylpolyhexamethylene adipamide), a cellulose ester (e.g., cellulose acetate, cellulose acetate succinate, cellulose acetate butyrate), a cellulose ether (e.g., methyl cellulose, ethyl cellulose, ethylbenzyl cellulose), a polycarbonate, a polyvinyl acetal (e.g., polyvinyl butyral, polyvinyl formal), a polyvinyl alcohol, a polyvinylpyrrolidone, acid-containing polymers and, copolymers disclosed in U.S. Pat. Nos. 3,458,311 and 4,273,857, and amphoteric polymer binders disclosed in U.S. Pat. No. 4,293,635. More preferred examples of the binder include a cellulose acetate butyrate polymer, a cellulose acetate lactate polymer, an acrylic polymer or interpolymer containing polymethyl methacrylate and methyl methacrylate/methacrylic acid and methyl methacrylate/acrylic acid copolymers, a terpolymer of methyl methacrylate/C2-C4 alkyl acrylate or methacrylate/acrylic or methacrylic acid, polyvinyl acetate, polyvinyl acetal, polyvinyl butyral, polyvinyl formal, and a mixture thereof.

A fluorine atom-containing polymer is also preferred as the low refractive index binder. The fluorine atom-containing polymer is preferably an organic solvent-soluble polymer containing, as the essential component, a fluoroolefin and containing, as the copolymerization component, one unsaturated monomer or two or more unsaturated monomers selected from an alkyl vinyl ether, an alicyclic vinyl ether, a hydroxy vinyl ether, an olefin, a haloolefin, an unsaturated carboxylic acid or an ester thereof, and a vinyl carboxylate. This polymer preferably has a mass average molecular weight of 5,000 to 200,000 and a fluorine atom content of 5 to 70 mass %.

Examples of the fluoroolefin in the fluorine atom-containing polymer include tetrafluoroethylene, chlorotrifluoroethylene, vinyl fluoride and vinylidene fluoride. Examples of the alkyl vinyl ether as the other copolymerization component include ethyl vinyl ether, isobutyl vinyl ether and n-butyl vinyl ether. Examples of the alicyclic vinyl ether include cyclohexyl vinyl ether and its derivatives. Examples of the hydroxy vinyl ether include hydroxybutyl vinyl ether. Examples of the olefin and haloolefin include ethylene, propylene, isobutylene, vinyl chloride and vinylidene chloride. Examples of the vinyl carboxylate include vinyl acetate and n-vinyl butyrate. Examples of the unsaturated carboxylic acid or an ester thereof include an unsaturated carboxylic acid such as (meth)acrylic acid and crotonic acid; C1-C18 alkyl esters of (meth)acrylic acid, such as methyl(meth)acrylate, ethyl(meth)acrylate, propyl(meth)acrylate, isopropyl(meth)acrylate, butyl(meth)acrylate, hexyl(meth)acrylate, octyl(meth)acrylate and lauryl(meth)acrylate; C2-C8 hydroxyalkyl esters of (meth)acrylic acid, such as hydroxyethyl(meth)acrylate and hydroxypropyl(meth)acrylate; an N,N-dimethylaminoethyl(meth)acrylate; and an N,N-diethylaminoethyl(meth)acrylate. One of these radical polymerizable monomers may be used alone, or two or more thereof may be used in combination. Furthermore, if desired, a part of the monomer may be replaced by another radical polymerizable monomer, for example, by a vinyl compound such as styrene, α-methylstyrene, vinyltoluene and (meth)acrylonitrile. Other monomer derivatives such as carboxylic acid group-containing fluoroolefin and glycidyl group-containing vinyl ether may also be used.

Specific examples of the above-described fluorine atom-containing polymer include “Lumifron” series having a hydroxyl group and being soluble in an organic solvent (for example, Lumifron LF200, weight average molecular weight: about 50,000, produced by Asahi Glass Company, Ltd.). In addition, organic solvent-soluble fluorine atom-containing polymers are commercially available from Daikin Kogyo Co., Ltd., Central Glass Co., Ltd., Penwalt and the like, and these also can be used.

Many of these binders form a non-three-dimensional crosslinked structure. The binder having a structure forming a three-dimensional crosslinked structure is described below.

(Binder that Forms Three-Dimensional Crosslinked Structure)

Many of the binders described above form a non-three-dimensional crosslinked structure, but in the recording material of the present invention, a binder having a structure forming a three-dimensional crosslinked structure may also be used. The binder having a structure forming a three-dimensional crosslinked structure is preferred in view of enhancing the film coatability, film strength and recording performance. Incidentally, the “binder having a structure forming a three-dimensional crosslinked structure” is referred to as a “matrix”.

The matrix contains a component forming its three-dimensional crosslinked structure, and this component for use in the present invention may contain a thermal crosslinking compound. As for the crosslinking component, a thermal crosslinking compound or a photocurable compound that is cured using a catalyst or the like and irradiating light, may be used, with a thermal crosslinking compound being preferred.

The thermal crosslinking matrix for use in the present invention is not particularly limited and may be appropriately selected according to the purpose, but examples thereof include a urethane resin formed from an isocyanate compound and an alcohol compound, an epoxy compound formed from an oxirane compound, and a polymer obtained by polymerizing a melamine compound, a formalin compound, an ester compound of unsaturated acid such as (meth)acrylic acid or itaconic acid, or an amide compound. Above all, a polyurethane matrix formed from an isocyanate compound and an alcohol compound is preferred and in consideration of recording preservability, a polyurethane matrix formed from a polyisocyanate and a polyalcohol is most preferred.

Specific examples of the polyisocyanate and polyalcohol which can form a polyurethane matrix are described below.

Specific examples of the polyisocyanate include biscyclohexylmethane diisocyanate, hexamethylene diisocyanate, phenylene-1,3-diisocyanate, phenylene-1,4-diisocyanate, 1-methoxyphenylene-2,4-diisocyanate, 1-methylphenylene-2,4-diisocyanate, 2,4-tolylene diisocyanate, 2,6-tolylene diisocyanate, 1,3-xylylene diisocyanate, 1,4-xylylene diisocyanate, biphenylene-4,4′-diisocyanate, 3,3′-dimethoxybiphenylene-4,4′-diisocyanate, 3,3′-dimethylbiphenylene-4,4′-diisocyanate, diphenylmethane-2,4′-diisocyanate, diphenylmethane-4,4′-diisocyanate, 3,3′-dimethoxydiphenylmethane-4,4′-diisocyanate, 3,3′-dimethyldiphenylmethane-4,4′-diisocyanate, naphthylene-1,5-diisocyanate, cyclobutylene-1,3-diisocyanate, cyclopentylene-1,3-diisocyanate, cyclohexylene-1,3-diisocyanate, cyclohexylene-1,4-diisocyanate, 1-methylcyclohexylene-2,4-diisocyanate, 1-methylcyclohexylene-2,6-diisocyanate, 1-isocyanate-3,3,5-trimethyl-5-isocyanatemethylcyclohexane, cyclohexane-1,3-bis(methylisocyanate), cyclohexane-1,4-bis(methylisocyanate), isophorone diisocyanate, dicyclohexylmethane-2,4′-diisocyanate, dicyclohexylmethane-4,4′-diisocyanate, ethylene diisocyanate, tetramethylene-1,4-diisocyanate, hexamethylene-1,6-diisocyanate, dodecamethylene-1,12-diisocyanate, phenyl-1,3,5-triisocyanate, diphenylmethane-2,4,4′-triisocyanate, diphenylmethane-2,5,4′-triisocyanate, triphenylmethane-2,4′,4″-triisocyanate, triphenylmethane-4,4′,4″-triisocyanate, diphenylmethane-2,4,2′,4′-tetraisocyanate, diphenylmethane-2,5,2′,5′-tetraisocyanate, cyclohexane-1,3,5-triisocyanate, cyclohexane-1,3,5-tris(methylisocyanate), 3,5-dimethylcyclohexane-1,3,5-tris(methylisocyanate), 1,3,5-trimethylcyclohexane-1,3,5-tris(methylisocyanate), dicyclohexylmethane-2,4,2′-triisocyanate, dicyclohexylmethane-2,4,4′-triisocyanatelysine diisocyanate methyl ester, and a prepolymer with isocyanate at both ends obtained by reacting such an organic isocyanate compound in excess of the stoichiometric amount with a polyfunctional active hydrogen-containing compound. Among these, biscyclohexylmethane diisocyanate and hexamethylene diisocyanate are preferred. One of these polyisocyanates may be used alone, or two or more thereof may be used in combination.

The polyalcohol may be a polyalcohol alone or a mixture with other polyalcohols. Examples of the polyalcohol include glycols such as ethylene glycol, triethylene glycol, diethylene glycol, polyethylene glycol, propylene glycol, polypropylene glycol and neopentyl glycol; diols such as butanediol, pentanediol, hexanediol, heptanediol and tetramethylene glycol; bisphenols or compounds obtained by modifying these polyalcohols with a polyethyleneoxy or polypropyleneoxy chain; glycerin; trimethylolpropane; and triols such as butanetriol, pentanetriol, hexanetriol and decanetriol or compounds obtained by modifying these polyalcohols with a polyethyleneoxy or polypropyleneoxy chain.

The content of the matrix-forming component in the optical recording composition using the dye monomer is preferably from 10 to 95 mass %, more preferably from 35 to 90 mass %.

[Material that Forms a Latent Image Capable of Creating a Refractive Index/Fluorescence Modulation Through Color Formation of a Dye]

The material that forms a latent image capable of creating a refractive index/fluorescence modulation through color formation of a dye include those containing a dye precursor capable of color formation upon oxidation reaction.

The dye precursor capable of color formation upon oxidation reaction is not particularly limited as long as it is a compound whose absorbance is increased by an oxidation reaction, but the dye precursor preferably contains at least one or more kinds of compounds selected from leuco quinone compounds, thiazine leuco compounds, oxazine leuco compounds, phenazine leuco compounds and leuco triarylmethane compounds.

Preferred examples of the leuco quinone compounds, thiazine leuco compounds, oxazine leuco compounds, phenazine leuco compounds and leuco triarylmethane compounds include the compounds described above, and these compounds may be used.

The material that forms a latent image capable of creating a refractive index/fluorescence modulation through color formation of a dye is described in more detail in JP-A-2005-320502.

[Material that Forms a Latent Image Capable of Creating a Refractive Index/Fluorescence Modulation Through Polymerization]

The material that forms a latent image capable of creating a refractive index/fluorescence modulation through polymerization is composed of:

1) a dye precursor capable of becoming a color former as a result of electron or energy transfer from the excited state of the two-photon absorption compound, the color former having absorption shifted to the longer wavelength side than in the original state and having absorption in the wavelength region where the molar absorption coefficient of linear absorption by the two-photon compound is 5,000 or less,

2) a polymerization initiator capable of initiating polymerization of a polymerizable compound as a result of electron or energy transfer from the excited state of the two-photon absorption compound,

3) a polymerizable compound, and

4) a binder.

The 2) polymerization initiator, 3) polymerizable compound and 4) binder are the same as those described above and therefore, in this section, “1) dye precursor capable of becoming a color former as a result of electron or energy transfer from the excited state of the two-photon absorption compound, the color former having absorption shifted to the longer wavelength side than in the original state and having absorption in the wavelength region where the molar absorption coefficient of linear absorption by the two-photon compound is 5,000 or less” (hereinafter sometimes simply referred to as a “dye precursor”) is described in detail.

The dye precursor of this section is preferably a dye precursor capable of becoming a color former having absorption shifted to the longer wavelength side than in the original state, as a result of direct electron or energy transfer from the excited state of the two-photon absorption compound or color former or by the action of an acid or base generated as a result of electron or energy transfer from the excited state of the two-photon absorption compound or color former to the acid or base generator.

In the two-photon absorption optical recording material using the dye precursor of this section, recording is preferably performed by a refractive index modulation. That is, when reading out the information, the color former preferably has no or almost no absorption at the readout light wavelength.

Accordingly, the dye precursor preferably becomes a color former having no absorption at the readout light wavelength and having absorption in the shorter wavelength side than that.

On the other hand, it is also preferred that even when the color former has absorption at the readout light wavelength, the color former decomposes in a step of exciting a latent image and thereby bringing about polymerization or in the subsequent fixing and loses its absorbing and sensitizing function.

As the dye precursor of this section, the following combinations are preferred:

A) a combination containing at least an acid-color forming dye precursor as the dye precursor and an acid generator and if desired, further containing an acid-increasing agent,

B) a combination containing at least a base-color forming dye precursor as the dye precursor and a base generator and if desired, further containing a base-increasing agent,

C) a combination containing a compound where an organic compound moiety having a function of cutting a covalent bond as a result of electron or energy transfer with the excited state of the two-photon absorption compound or color former is covalently bonded with an organic compound moiety characterized by becoming a color former when covalently bonded and when released, or further containing a base, and

D) a combination containing a compound capable of undergoing a reaction as a result of electron transfer with the excited state of two-photon absorption compound or color former and thereby changing the absorption form.

In any of these cases, when an energy transfer mechanism from the excited state of the two-photon absorption compound or color former is used, the mechanism may be either a Forster mechanism where energy transfer occurs from a singlet excited state of the two-photon absorption compound or color former, or a Dexter mechanism where energy transfer occurs from a triplet excited state.

At this time, in order to cause the energy transfer with good efficiency, the excitation energy of the two-photon absorption compound or color former is preferably larger than the excitation energy of the dye precursor.

In the case of an electron transfer mechanism from the excited state of the two-photon absorption compound or color former, the mechanism may be either a mechanism where electron transfer occurs from a singlet excited state of the two-photon absorption compound or color former, or a mechanism where electron transfer occurs from a triplet excited state.

The excited state of the two-photon absorption compound or color former may give an electron to the dye precursor, acid generator or base generator or may receive an electron. In the case of giving an electron from the excited state of the two-photon absorption compound or color former, in order to cause the electron transfer with good efficiency, the orbital (LUMO) where an excited electron is present in the excited state of the two-photon absorption compound or color former preferably has a higher energy than the energy of the LUMO orbital of the dye precursor or acid or base generator.

In the case where the excited state of the two-photon absorption compound or color former receives an electron, in order to cause the electron transfer with good efficiency, the orbital (HOMO) where a hole is present in the excited state of the two-photon absorption compound of color former preferably has a lower energy than the energy of the HOMO orbital of the dye precursor or acid or base generator.

The preferred combination of the dye precursor is described in detail below. The case where the dye precursor is an acid-color forming dye precursor and further contains an acid generator is described below.

At this time, the acid generator is a compound capable of generating an acid as result of energy or electron transfer from the excited state of the two-photon absorption compound or color former. The acid generator is preferably stable in a dark place. The acid generator for use in this section is preferably a compound capable of generating an acid as a result of electron transfer from the excited state of the two-photon absorption compound or color former.

As for the acid generator used in this section, the following 6 systems are preferred. Preferred examples thereof are the same as those described above for the cationic polymerization initiator.

That is, 1) a trihalomethyl-substituted triazine-based acid generator, 2) a diazonium salt-based acid generator, 3) a diaryliodonium salt-based acid generator, 4) a sulfonium salt-based acid generator, 5) a metal arene complex-based acid generator, and 6) a sulfonic acid ester-based acid generator are preferred, and 3) a diaryliodonium salt-based acid generator, 4) a sulfonium salt-based acid generator, and 6) a sulfonic acid ester-based acid generator are more preferred.

In the case of simultaneously using cationic polymerization and an acid-color forming precursor, the functions of the cationic polymerization initiator and the acid generator are preferably fulfilled by the same compound. These acid generators may be used as a mixture of two or more thereof in an arbitrary ratio, if desired.

The acid-color forming dye precursor in the case where the dye precursor used in the two-photon absorption optical recording material of the present invention is an acid-color forming dye precursor and further contains an acid generator, is described below.

The acid-color forming dye precursor for use in this section is a dye precursor capable of becoming a color former having absorption changed from the original state, by the action of an acid generated from the acid generator. The acid-color forming dye precursor for use in this section is preferably a compound whose absorption is shifted to a longer wavelength side by the action of an acid, more preferably a compound which is colorless but color-formed by the action of an acid.

Preferred examples of the acid-color forming dye precursor include a triphenylmethane-based compound, a phthalide-based compound (including indolylphthalide-based, azaphthalide-based and triphenylmethane phthalide-based compounds), a phenothiazine-based compound, a phenoxazine-based compound, a fluorane-based compound, a thiofluorane-based compound, a xanthene-based compound, a diphenylmethane-based compound, a chromenopyrazole-based compound, leucoauramine, a methine-based compound, a azomethine-based compound, a Rhodamine lactam-based compound, a quinazoline-based compound, a diazaxanthene-based compound, a fluorene-based compound and a spiropyran-based compound. The acid-color forming dye precursor is more preferably a leuco dye having a partial structure such as lactone, lactam, oxazine and spiropyran, and examples thereof include fluorane-based, thiofluorane-based, phthalide-based, Rhodamine lactam-based and spiropyran-based compounds. Specific examples of these compounds are disclosed, for example, in JP-A-2002-156454, patents cited therein, JP-A-2000-281920, JP-A-11-279328 and JP-A-8-240908.

The dye produced from the acid-color forming dye precursor for use in this section is preferably a xanthene dye, a fluorane dye or a triphenylmethane dye.

These acid-color forming dye precursors may be used as a mixture of two or more thereof in an arbitrary ratio, if desired.

Specific preferred examples of the acid-color forming dye precursor for use in the present invention include the compounds described above, and these compounds may be used.

In the case where the dye precursor group for use in this section contains at least an acid-color forming dye precursor as the dye precursor and an acid generator, the dye precursor group may further contain an acid-increasing agent.

The acid-increasing agent is a compound capable of increasing an acid by using, as a trigger, a small amount of an acid generated from the acid generator in such a way that the acid-increasing agent is stable in the absence of an acid but decomposes in the presence of an acid to release an acid and another acid-increasing agent is decomposed by the action of the acid to again release an acid.

Preferred examples of the acid-increasing agent include compounds having a structure represented by formulae (34-1) to (34-6) of JP-A-2005-97538. More preferred specific examples thereof include compounds described in paragraphs 0299 to 0301 of the same patent publication.

The system is preferably heated at the time of increasing an acid and therefore, is preferably heat-treated in a step of exciting a latent image and thereby bringing about polymerization or in a separately provided fixing step.

The case where the dye precursor is a base-color forming dye precursor and further contains a base generator is described below.

The base generator is a compound capable of generating a base as a result of energy or electron transfer from the excited state of the two-photon absorption compound or color former. The base generator is preferably stable in a dark place. The base generator for use in this section is preferably a compound capable of generating a base as a result of electron transfer from the excited state of the two-photon absorption compound or color former.

The base generator for use in this section preferably generates a Broensted base by the action of light, more preferably generates an organic base, still more preferably generates an amine as the organic base.

Preferred examples of the base generator in the dye precursor for use in this section are the same as those of the base generator for the above-described anionic polymerization initiator.

In the case of simultaneously using anionic polymerization and a base-color forming precursor, the functions of the anionic polymerization initiator and the base generator are preferably fulfilled by the same compound.

These base generators may be used as a mixture of two or more thereof in an arbitrary ratio, if desired.

The base-color forming dye precursor when the dye precursor for use in this section is a base-color forming dye precursor and further contains a base generator, is described below.

The base-color forming dye precursor for use in this section is a dye precursor capable of becoming a color former having absorption changed from the original state, by the action of a base generated from the base generator.

The base-color forming dye precursor for use in this section is preferably a compound whose absorption is shifted to the longer wavelength side by the action of a base, more preferably a compound which is colorless but color-formed by the action of a base.

Specific preferred examples of the base-color forming dye precursor for use in this section include the compounds described above, and those compounds may be used.

In the case where the dye precursor for use in this section is a base-color forming dye precursor, the dye precursor may further contain a base-increasing agent in addition to the base generator.

The base-increasing agent is a compound capable of increasing a base by using, as a trigger, a small amount of a base generated from the base generator in such a way that the base-increasing agent is stable in the absence of a base but decomposes in the presence of a base to release a base and another base-increasing agent is decomposed by the action of the base to again release a base.

Examples of the base-increasing agent include compounds of structures represented by formulae (34-1) to (34-6) and set forth in paragraph 0287 of JP-A-2005-97538. More preferred specific examples thereof include compounds described in paragraphs 0299 to 0301 of the same patent publication.

The system is preferably heated at the time of increasing a base and therefore, in the case of using a base-increasing agent, the system is preferably heat-treated in a step of exciting a latent image and thereby bringing about polymerization or in a separately provided fixing step.

The case where the dye precursor for use in this section is a compound where an organic compound moiety having a function of cutting a covalent bond as a result of electron or energy transfer with the excited state of the two-photon absorption compound or color former is covalently bonded with an organic compound moiety characterized by becoming a color former when covalently bonded and when released is described below.

Examples of the compound which can be used in this section include compounds represented by formula (32), more specifically, compounds of structures set forth in paragraphs of 0326 to 0348, of JP-A-2005-97538.

The two-photon absorption optical recording material of the present invention preferably further contains a base, if desired, for the purpose of dissociating the produced dissociative dye. The base may be either an organic base or an inorganic base, and preferred examples thereof include alkylamines, anilines, imidazoles, pyridines, carbonates, hydroxide salts, carboxylates and metal alkoxide. Also, polymers containing such a base are preferably used.

The case where the dye precursor for use in this section is a compound capable of undergoing a reaction as a result of electron transfer with the excited state of the two-photon absorption compound or color former and thereby changing the absorption form is described below. The compounds capable of causing such a change are referred to by a generic term of so-called “electrochromic compound”.

Preferred examples of the electrochromic compound for use as the dye precursor in this section include polypyrroles (preferably, for example, polypyrrole, poly(N-methylpyrrole), poly(N-methylindole) and polypyrrolopyrrole), polythiophenes (preferably, for example, polythiophene, poly(3-hexylthiophene), polyisothianaphthene, polydithienothiophene and poly(3,4-ethylenedioxy)thiophene), polyanilines (preferably, for example, polyaniline, poly(N-naphthylaniline), poly(o-phenylenediamine), poly(aniline-m-sulfonic acid), poly(-methoxyaniline) and poly(o-aminophenol)), a poly(diarylamine), a poly(N-vinylcarbazole), a Co pyridinoporphyrazine complex, an Ni phenanthroline complex and an Fe basophenanthroline complex.

In addition, electrochromic materials such as viologens, polyviologens, lanthanoid diphthalocyanines, styryl dyes, TNFs, TCNQ/TTF complexes, Ru trisbipyridyl complexes are also preferred.

In the case where the dye precursor is a compound capable of undergoing a reaction as a result of electron transfer with the excited state of the two-photon absorption compound or color former and thereby changing the absorption form, the dye precursor for use in this section is preferably at least a compound represented by formula (37), more specifically, a compound of a structure set forth in paragraphs 0352 to 0352, of JP-A-2005-97538. Specific preferred examples thereof include compounds described in paragraph 0354 of the same patent publication.

The dye precursor for use in this section may be a commercially available product or may be synthesized by a known method.

In the two-photon absorption optical recording material of the present invention, an electron-donating compound having an ability of reducing the radical cation of the two-photon absorption compound or color former, or an electron-accepting compound having an ability of oxidizing the radical anion of the two-photon absorption compound or color former can be preferably used. In particular, use of an electron-donating compound is more preferred in view of enhancing the coloring speed.

Preferred examples of the electron-donating compound for use in the present invention include compounds described in paragraph 0357 of JP-A-2005-97538, and compounds set forth above as examples of usable compounds in [Material that creates a refractive index or fluorescence modulation through color formation of a dye or a fluorescent dye]. On the other hand, preferred examples of the electron-accepting compound for use in the present invention include compounds described in paragraph 0358 of the same patent publication and compounds set forth in paragraphs 2022 to 0212 of JP-A-2007-87532.

The oxidation potential of the electron-donating compound is preferably baser (on the minus side) than the oxidation potential of the two-photon absorption compound or color former or than the reduction potential of the excited state of the two-photon absorption compound or color former, and the reduction potential of the electron-accepting compound is preferably nobler (on the plus side) than the reduction potential of the two-photon absorption compound or color former or than the oxidation potential of the excited state of the two-photon absorption compound or color former.

The material that forms a latent image capable of creating a refractive index/fluorescence modulation through polymerization is described in more detail in JP-A-2005-97538.

### [Other Components]

The two-photon absorption optical recording material of the present invention may further use a binder. The polymer matrix for use in the polymer composition of the present invention is not particularly limited and may be an organic polymer compound or an inorganic polymer compound. The organic polymer compound is preferably a solvent-soluble thermoplastic polymer, and one polymer may be used alone or some polymers may be used in combination. A thermoplastic polymer well compatible with various components dispersed in the polymer composition is preferred.

As for the binder used in the recording material of the present invention, those set forth as preferred examples of usable binders in the item of [Material that creates a refractive index modulation through polymerization of a dye having a polymerizable group] all can be used. Other specific examples include compounds described in paragraph 0022 (an acrylate, an α-alkyl acrylate ester, an acidic polymer, an interpolymer, a polyvinyl ester, an ethylene/vinyl acetate copolymer, a saturated or unsaturated polyurethanes, a butadiene or isoprene polymer or copolymer, a high molecular weight polyethylene oxide of polyglycol, an epoxy compound, a cellulose ester, a cellulose ether, a polycarbonate, a norbornene-based polymer, a polyvinyl acetal, a polyvinyl alcohol, a polyvinylpyrrolidone) of JP-A-2005-320502; and a polystyrene polymer or copolymer, a polymer produced from a reaction product of a copolyester polymethylene glycol and an aromatic acid compound or a mixture thereof, a poly-N-vinyl carbazole or a copolymer thereof, and a carbazole-containing polymer described in the same paragraph. Also, specific preferred examples of the binder include fluorine atom-containing polymers described in paragraphs 0023 to 0024 of the same patent publication.

The binder for use in the present invention is preferably, an acrylate, an α-alkyl acrylate ester, a polystyrene, a polyalkylsytrene or a polystyrene copolymer, and in view of enhancing detection sensitivity, more preferably an acrylate, an α-alkyl acrylate, a polystyrene or a polystyrene copolymer. As for specific examples thereof, examples of the acrylate and α-alkyl acrylate ester include methyl(meth)acrylate, ethyl(meth)acrylate, propyl(meth)acrylate, butyl(meth)acrylate, isobutyl(meth)acrylate, pentyl(meth)acrylate, hexyl(meth)acrylate, octyl(meth)acrylate, 2-ethylhexyl(meth)acrylate, lauryl(meth)acrylate, stearyl(meth)acrylate and cyclohexyl(meth)acrylate; and examples of the (meth)acrylate having a benzene ring include benzyl(meth)acrylate, phenoxyethyl(meth)acrylate, phenoxypolyethylene glycol(meth)acrylate and nonylphenol ethylene oxide adduct (meth)acrylate. In particular, benzyl(meth)acrylate and phenoxyethyl(meth)acrylate are preferred as the (meth)acrylate having a benzene ring. Only one kind of such a monomer may be used, or two or more kinds thereof may be used in combination. In the (meth)acrylate-based copolymer, other copolymerizable monomers copolymerizable with an alkyl(meth)acrylate, a benzene ring-containing (meth)acrylate or a nitrogen-containing radical polymerizable monomer may be copolymerized, and examples of the other copolymerizable monomer include alkyl vinyl ethers such as allyl glycidyl ether, methyl vinyl ether, ethyl vinyl ether, isobutyl vinyl ether, n-butyl vinyl ether, 2-ethyl hexyl vinyl ether, n-octyl vinyl ether, lauryl vinyl ether, cetyl vinyl ether and stearyl vinyl ether, alkoxyalkyl(meth)acrylates such as methoxyethyl(meth)acrylate and butoxyethyl(meth)acrylate, glycidyl(meth)acrylate, vinyl acetate, vinyl propionate, (anhydrous) maleic acid, acrylonitrile and vinylidene chloride. A compound having a hydrophilic polar group may also be copolymerized, and examples of the polar group include —SO₃M, —PO(OM)₂ and —COOM (wherein M represents a hydrogen atom, an alkali metal or ammonium).

Examples of the polyalkylstyrene compound include polymethylstyrene, polyethylstyrene, polypropylstyrene, polybutylstyrene, polyisobutylstyrene, polypentylstyrene, hexylpolystyrene, polyoctylstyrene, poly-2-ethylhexylstyrene, polylaurylstyrene, polystearylstyrene and polycyclohexylstyrene; and examples of the (meth)acrylate having a benzene ring include polybenzylstyrene, polyphenoxyethylstyrene, polyphenoxy polyethylene glycol styrene and polynonylphenolstyrene. The position of the alkyl is preferably the α- or para-position. Only one kind of such a monomer may be used, or two or more kinds thereof may be used in combination. In the polystyrene copolymer, other copolymerizable monomers copolymerizable with a conjugated diene compound, an alkylstyrene, a benzene ring-containing styrene or a nitrogen-containing radical polymerizable monomer may be copolymerized, and examples of the other copolymerizable monomer include acetylene, butadiene, acrylonitrile, vinylidene chloride, polyethylene, allyl glycidyl ether, methyl vinyl ether, ethyl vinyl ether, isobutyl vinyl ether, n-butyl vinyl ether, 2-ethylhexyl vinyl ether, n-octyl vinyl ether, lauryl vinyl ether, cetyl vinyl ether and stearyl vinyl ether.

In the two-photon absorption optical recording material of the present invention, a heat stabilizer may be added for the purpose of enhancing the storability during storage.

Useful examples of the heat stabilizer include hydroquinone, phenidone, p-methoxyphenol, alkyl or aryl-substituted hydroquinone or quinone, catechol, tert-butyl catechol, pyrogallol, 2-naphthol, 2,6-di-tert-butyl-p-cresol, phenothiazine and chloranil. Also, dinitroso dimers described in U.S. Pat. No. 4,168,982 by Pazos are useful.

In the two-photon absorption optical recording material of the present invention, a plasticizer may be used for varying the adhesion, flexibility, hardness and other various mechanical properties of the optical recording material. Examples of the plasticizer include triethylene glycol dicaprylate, triethylene glycol bis(2-ethylhexanoate), tetraethylene glycol diheptanoate, diethyl sebacate, dibutyl suberate, tris(2-ethylhexyl)phosphate, tricresyl phosphate and dibutyl phthalate.

The two-photon absorption optical recording material of the present invention may be prepared by an ordinary method, for example, by adding the above-described essential components and arbitrary components with or without a solvent.

Examples of the solvent include a ketone-based solvent such as methyl ethyl ketone, methyl isobutyl ketone, acetone and cyclohexanone, an ester-based solvent such as ethyl acetate, butyl acetate, ethylene glycol diacetate, ethyl lactate and cellosolve acetate, a hydrocarbon-based solvent such as cyclohexane, toluene and xylene, an ether-based solvent such as tetrahydrofuran, dioxane and diethyl ether, a cellosolve-based solvent such as methyl cellosolve, ethyl cellosolve, butyl cellosolve and dimethyl cellosolve, an alcohol-based solvent such as methanol, ethanol, n-propanol, 2-propanol, n-butanol and diacetone alcohol, a fluorine-based solvent such as 2,2,3,3-tetrafluoropropanol, a halogenated hydrocarbon-based solvent such as dichloromethane, chloroform and 1,2-dichloroethane, an amide-based solvent such as N,N-dimethylformamide, and a nitrile-based solvent such as acetonitrile and propionitrile.

The two-photon absorption optical recording material of the present invention may be directly coated on a substrate by using a spin coater, a roll coater, a bar coater or the like, or may be cast as a film and then laminated on a substrate by an ordinary method, whereby a two-photon absorption optical recording material can be obtained.

The term “substrate” as used herein means an arbitrary natural or synthetic support, suitably a material which can be present in the form of a soft or rigid film, a sheet or a plate.

Preferred examples of the substrate include polyethylene terephthalate, resin-undercoated polyethylene terephthalate, polyethylene terephthalate subjected to flame or electrostatic discharge treatment, cellulose acetate, polycarbonate, polymethyl methacrylate, polyester, polyvinyl alcohol and glass.

The solvent used can be removed by evaporation at the drying. For the removal by evaporation, heating or reduced pressure may be used.

Furthermore, a protective layer for blocking oxygen may be formed on the two-photon absorption optical recording material. The protective layer may be laminated, for example, by stacking a plastic-made film or sheet such as polyolefin (e.g., polypropylene, polyethylene), polyvinyl chloride, polyvinylidene chloride, polyvinyl alcohol, polyethylene terephthalate or cellophane film, with use of electrostatic adhesion or an extruder or may be formed by coating a solution of the polymer above. Also, a glass sheet may be laminated. Between the protective layer and the photosensitive film and/or between the substrate and the photosensitive film, a pressure-sensitive adhesive or a liquid substance may be allowed to be present so as to increase the air tightness.

Furthermore, the two-photon absorption optical recording material of the present invention may have a multilayer structure where a recording layer containing recording components and a non-recording layer containing no recording components are alternately stacked. By virtue of having a structure where a recording layer and a non-recording layer are alternately stacked, a non-recording layer intervenes between recording layers and blocks expansion of the recording region in a direction perpendicular to the recording layer surface. Accordingly, even when the recording layer is restricted to a thickness on the order of irradiation light wavelength, crosstalk can be reduced. As a result, not only the thickness of the recording layer itself can be made thin but also the interlayer distance of recording layers including a non-recording layer can be shortened.

The thickness of the recording layer is preferably in the range from 50 to 5,000 nm, more preferably from 100 to 1,000 nm, still more preferably from 100 to 500 nm, according to the amount of refractive index change of the recording layer material used, because the amount of refractive index change of the recording layer during recording and the interference conditions by reflected light on the front and back surfaces of each recording layer with respect to the incident direction of light need to be satisfied.

The non-recording layer is a thin-film like layer formed from a material that causes no change in the absorption spectrum or light emission spectrum when recording light is irradiated.

The material used for the non-recording layer is preferably a material dissolvable in a solvent incapable of dissolving the material used for the recording layer, because the production involving the formation of a multilayer structure is easy. Above all, a transparent polymer material not having absorption in the visible light region is preferred. A water-soluble polymer is suitably used as such a material.

Specific examples of the water-soluble polymer include polyvinyl alcohol (PVA), polyvinylpyridine, polyethyleneimine, polyethylene oxide, polypropylene oxide, polyvinylpyrrolidone, polyacrylamide, polyacrylic acid, sodium polyacrylate, carboxymethyl cellulose, hydroxyethyl cellulose and gelatin. Among these, PVA, polyvinylpyridine, polyacrylic acid, polyvinylpyrrolidone, carboxymethyl cellulose and gelatin are preferred, and PVA is most preferred.

In the case of using a water-soluble polymer as the material for the non-recording layer, a coating solution obtained by dissolving the water-soluble polymer in water is coated, for example, by a coating method such as spin coating, whereby the non-recording layer can be formed.

The thickness of the non-recording layer is preferably in the range from 1 to 50 μm, more preferably from 1 to 20 μm, still more preferably from 1 to 10 μm, in view of wavelength of recording or readout light, recording power, readout power, NA of lens and recording sensitivity of the recording layer material, so as to reduce the crosstalk between recording layers sandwiching the non-recording layer.

The number of pairs of recording layers and non-recording layers stacked alternately is preferably in the range from 9 to 200, more preferably from 10 to 100, still more preferably from 10 to 30, in view of recording capacity required of the two-photon absorption recording medium and aberration determined by the optical system used.

### Examples

Working examples of the present invention are specifically described below based on experimental results. Of course, the present invention is not limited to these working examples.

### Example 1

### <Synthesis Method of Compound D-1>

Compound D-1 was synthesized by the following method.

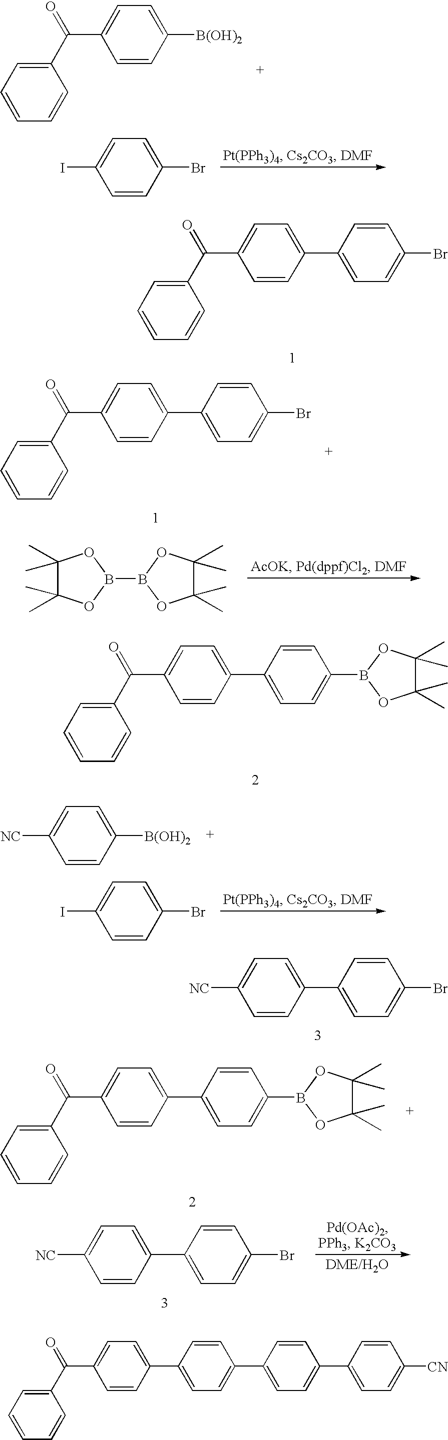

### Synthesis of Raw Material Compound 1:

After dissolving 2.7 g (12 mmol) of 4-benzoylphenylboronic acid and 2.8 g (10 mmol) of 1-bromo-4-iodobenzene in 50 ml of dimethylformamide (DMF), 0.6 g (0.5 mmol) of tetrakis(triphenylphosphine)platinum and 6.5 g (20 mmol) of cesium carbonate were added thereto, and the resulting solution was heated for 8 hours under a nitrogen flow.

The reaction solution was allowed to cool and extracted by adding distilled water and about 600 ml of ethyl acetate, and the organic layer was separated by removing the aqueous layer and dried over magnesium sulfate. The magnesium sulfate was removed by filtration, and the filtrate was evaporated to dryness by a rotary evaporator and purified through a silica gel column (ethyl acetate:hexane=1:10) to obtain 1.6 g (yield: 48%) of colorless Raw Material Compound 1. Compound 1 obtained was confirmed to be the objective compound by mass spectrum and 1H NMR spectrum.

### Synthesis of Raw Material Compound 2:

After suspending 0.68 g (2 mmol) of Raw Material Compound 1, 0.63 g (2.5 mmol) of bis(pinacolate)diboron, 0.59 g (6 mmol) of potassium acetate and 100 mg (0.12 mmol) of [1,1′-bis(diphenylphosphino)ferrocene]dichloropalladium in 50 ml of DMF, the solution was heated at 80° C. for 9 hours under a nitrogen flow. The reaction solution was allowed cool and extracted by adding distilled water and ethyl acetate, the organic layer was separated by removing the aqueous layer and dried over magnesium sulfate. The magnesium sulfate was removed by filtration, and the filtrate was evaporated to dryness by a rotary evaporator and purified through a silica gel column (ethyl acetate:hexane=1:20) to obtain 0.65 g (yield: 85%) of colorless Raw Material Compound 2. Compound 2 obtained was confirmed to be the objective compound by mass spectrum and 1H NMR spectrum.

### Synthesis of Raw Material Compound 3:

After dissolving 1.76 g (12 mmol) of 4-cyanobenzeneboronic acid and 2.8 g (10 mmol) of 1-bromo-4-iodobenzene in 60 ml of DMF, 0.6 g (0.5 mmol) of tetrakis(triphenylphosphine)platinum and 6.5 g (20 mmol) of cesium carbonate were added thereto, and the resulting solution was heated at 120° C. for 8 hours under a nitrogen flow. The reaction solution was allowed to cool and extracted by adding distilled water and about 600 ml of ethyl acetate, and the organic layer was separated by removing the aqueous layer and dried over magnesium sulfate. The magnesium sulfate was removed by filtration, and the filtrate was evaporated to dryness by a rotary evaporator and purified through a silica gel column (ethyl acetate:hexane=1:10) to obtain 0.53 g (yield: 21%) of colorless Raw Material Compound 3. Compound 3 obtained was confirmed to be the objective compound by mass spectrum and 1H NMR spectrum.

### Synthesis of Compound D-1:

After dissolving 0.5 g (1.3 mmol) of Raw Material Compound 2 and 0.33 g (1.3 mmol) of Raw Material Compound 3 in a mixed solvent of 20 ml of distilled water and 14 ml of ethylene glycol dimethyl ether, 14.6 mg (0.065 mmol) of palladium acetate, 34 mg (0.13 mmol) of triphenylphosphine and 0.97 g (7 mmol) of potassium carbonate were added thereto, and the resulting solution was refluxed under heating for 2 hours. The reaction solution was allowed to cool and extracted by adding distilled water and dichloromethane, and the organic layer was separated by removing the aqueous layer and dried over magnesium sulfate. The magnesium sulfate was removed by filtration, and the filtrate was evaporated to dryness by a rotary evaporator to obtain a crude product. The obtained crude produce was purified by sublimation to obtain 0.11 g (yield: 19%) of the objective. The compound obtained was confirmed to be the objective Compound D-1 by mass spectrum and 1H NMR spectrum.

¹H NMR (CDCl₃): 7.52 (t, 2H), 7.62 (t, 1H), 7.71 (d, 2H), 7.78 (m, 12H), 7.86 (d, 2H), 7.93 (d, 2H).

### <Measuring Method of Two-Photon Absorption Cross-Sectional Area>

The measurement of the two-photon absorption cross sectional area of the synthesized compound was performed according to the Z-scanning method described in Mansoor Sheik-Bahae et al., IEEE. Journal of Quantum Electronics, 26, 760 (1990). The Z-scanning method is a method widely utilized as a measuring method of a nonlinear optical constant, where a measurement sample is moved along a beam in the vicinity of the focal point of converged laser beams and the change of the quantity of transmitted light is recorded. The power density of incident light varies depending on the position of the sample and therefore, in the case where there is nonlinear absorption, the quantity of transmitted light attenuates near the focal point. The two-photon absorption cross-sectional area was computed by fitting the change of the quantity of transmitted light to a theoretical curve estimated from the incident light intensity, converging spot size, thickness and concentration of the sample, and the like. In the measurement of the two-photon absorption cross-sectional area, a Ti:sapphire pulsed laser (pulse width: 100 fs, repetition: 80 MHz, average output: 1 W, peak power: 100 kW) was used as the light source. A solution obtained by dissolving the compound in chloroform in a concentration of 1×10⁻³ was used as the sample for the measurement of two-photon absorption.

### <Evaluation of Two-Photon Absorption Cross-Sectional Area>

Two-photon absorption cross-sectional areas of Compound D-1 of the present invention and Compound R-1 (Comparative Compound R-1 shown below) described in Y. Morel, O. Stephan, C. Andraud, and P. L. Baldeck, Synth. Met., 2001, 124, 237 are shown in Table 1.

TABLE 1
Two-Photon Absorption Cross-Sectional Area

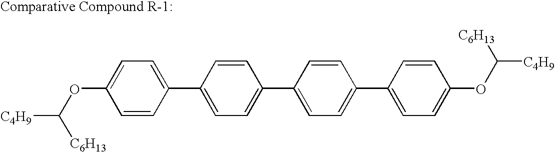

| | Two-Photon Absorption | Two-Photon Absorption | |
|---|---|---|---|
| Compound | Cross-Sectional Area/GM | Maximum Wavelength/nm | |
| D-1 | 200 | 550 | Invention |
| R-1 | 15 | near 525 | Comparative Example |
| | | | (value in document) |

1 GM = 1 × 10⁻⁵⁰ cm⁴ s molecule⁻¹ photon⁻¹

### <Preparation of Two-Photon Recording Material>

### (Preparation of Two-Photon Absorption Recording Material 1)

Two-Photon Absorption Recording Material 1 was prepared according to the following formulation.

| | | |
|---|---|---|
| Two-Photon absorption compound: D-1 | 1.0 | parts by mass |
| Dye precursor: DP-1 | 5.0 | parts by mass |
| Acid generator: PAG-1 | 5.0 | parts by mass |
| Binder: polyvinyl acetate (produced by Aldrich, | 100 | parts by mass |
| Mw = 113,000) | | |
| Coating solvent: dichloromethane | 2800 | parts by mass |

### (Preparation of Two-Photon Absorption Recording Material 2)

Two-Photon Absorption Recording Material 2 was prepared in the same manner as Two-Photon Absorption Recording Material 1 except for using D-6 in place of D-1 as the two-photon absorption compound.

### (Preparation of Two-Photon Absorption Recording Material 3)

Two-Photon Absorption Recording Material 3 was prepared according to the following formulation.

| | | |
|---|---|---|
| Two-Photon absorption compound: D-1 | 1.0 | parts by mass |
| Dye precursor: Lo-11 | 3.3 | parts by mass |
| Binder: poly(methyl methacrylate-co-ethyl | 66.7 | parts by mass |
| acrylate) (produced by Aldrich, | | |
| Mw = 101,000) | | |
| Coating solvent: dichloromethane | 1,400 | parts by mass |

### (Preparation of Two-Photon Absorption Recording Material 4)

Two-Photon Absorption Recording Material 4 was prepared according to the following formulation.

| | | |
|---|---|---|
| Two-Photon absorption compound: D-1 | 1.0 | parts by mass |
| Monomer: M-1 | 92 | parts by mass |
| Polymerization initiator: I-1 | 2.0 | parts by mass |
| Binder: cellulose acetate butyrate (produced | 100 | parts by mass |
| by Eastman Chemical Company, CAB531-1) | | |
| Coating solvent: dichloromethane | 2,900 | parts by mass |

### (Preparation of Two-Photon Absorption Recording Material 1 for Comparison (Comparative Material 1))

Comparative Material 1 was prepared in the same manner as Two-Photon Absorption Recording Material 1 except for using Two-Photon Absorption Compound D-104 described in JP-A-2007-87532 in place of D-1 as the two-photon absorption compound of Two-Photon Absorption Recording Material 1.

### (Preparation of Two-Photon Absorption Recording Material 2 for Comparison (Comparative Material 2))

Comparative Material 2 was prepared in the same manner as Two-Photon Absorption Recording Material 4 except for using Two-Photon Absorption Compound D-104 described in JP-A-2007-87532 in place of D-1 as the two-photon absorption compound of Two-Photon Absorption Recording Material 4.

The dye precursor: DP1, acid generator: PAG-1, dye precursor: Lo-11, monomer: M-1 and polymerization initiator I-1 used are as follows.

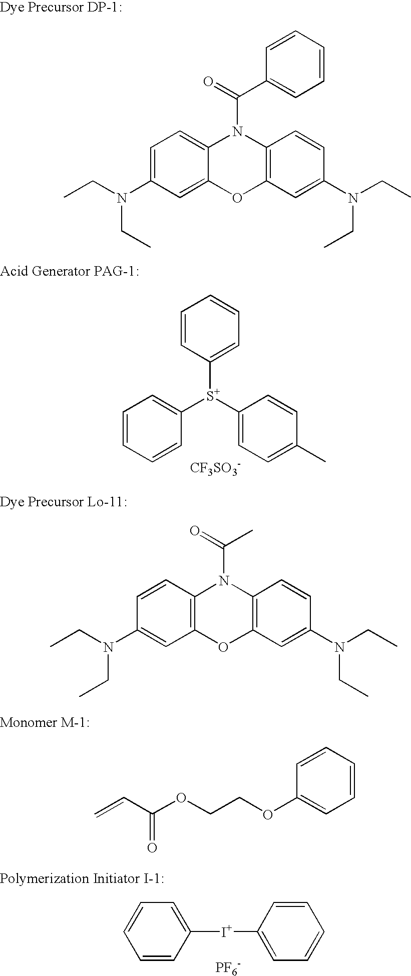

### <Production of Two-Photon Absorption Recording Medium>

The two-photon absorption recording medium of the present invention was produced as a thin film having a coating thickness of 1 μm after drying by spin-coating each of Two-Photon Absorption Recording Materials 1 to 4 on a slide glass. Incidentally, the mediums for comparison were also produced similarly by a spin coating method. The recording medium obtained from Two-Photon Absorption Recording Material 1 was designated as Two-Photon Absorption Recording Medium 1. The same applies to other recording mediums.

### <Evaluation of Two-Photon Absorption Performance>

Second harmonic 522 nm of a femtosecond laser at 1045 nm (pulse width: 200 fs, repetition: 2.85 GHz, peak power: 1 kW) was used for the two-photon recording. When reading out the recording signal, a fluorescence signal generated by the irradiation of He—Ne laser light at 632 nm was read out in the case of a fluorescence-modulating material (Two-Photon Absorption Recording Materials 1 and 2 and Comparative Material 1), and a reflected light signal by the irradiation of semiconductor laser light at 405 nm was read out in the case of a refractive index-modulating material (Two-Photon Absorption Recording Material 3 and Comparative Material 2). For the judgement whether two-photon recording or not, the recording light intensity dependency of the readout signal was measured and when the signal intensity was proportional to the square of the recording light intensity, this was judged that recording by two-photon absorption was performed (evaluation of quadratic dependency). The results are shown in Table 2 below.

TABLE 2
<Evaluation Results of Two-Photon Recording Performance>

| | Recording Light | Presence or | |
|---|---|---|---|
| | Intensity | Absence of | |
| | Dependency of | Quadratic | |
| Recording Medium | Readout Signal | Dependency | Judgment |
| Two-Photon Recording Medium 1 | quadratic | present | two-photon recording |
| Two-Photon Recording Medium 2 | quadratic | present | two-photon recording |
| Two-Photon Recording Medium 3 | quadratic | present | two-photon recording |
| Comparative Medium 1 | linear | none | one-photon recording |
| Two-Photon Recording Medium 4 | quadratic | present | two-photon recording |
| Comparative Medium 2 | linear | none | one-photon recording |

Two-Photon recording could not be performed when Two-Photon Absorption Compound D-104 described in JP-A-2007-87532 was used, because this compound has linear absorption at the two-photon recording wavelength 522 nm used in the present invention.

### [Characteristic Evaluation of Two-Photon Absorption Compound by Computation]

The two-photon absorption cross-sectional area of the compound was predicted by computing the values of the following mathematical formulae (1) and (2).

$$δ ( ω ) = ( \frac{3 π {hv}^{2}}{{n}^{2} {c}^{2} {ɛ}_{0}} ) Im γ ( - ω ; ω , - ω , ω ) \qquad Mathematical Formula ( 1 )$$

wherein c: light speed, ν: frequency, n: refractive index, ε₀: dielectric constant in vacuum, ω: vibration frequency, and Im: imaginary part. The imaginary part (Imγ) of γ has the following relationship with Mge: dipole moment between |g> and |e>, Mge′: dipole moment between |g> and |e′>, Δμge: different of dipole moment between |g> and |e>, Ege: transition energy, and Γ: damping factor.

$$Im γ ( - ω ; ω , - ω , ω ) = Im P [ \begin{matrix}\begin{matrix}\frac{{Mge}^{2} Δμ {ge}^{2}}{\begin{matrix}\begin{matrix}( Ege - ℏω - iΓ ge ) \\ ( Ege - 2 ℏω - iΓ ge )\end{matrix} \\ ( Ege - ℏω - iΓ ge )\end{matrix}} + \\ {\sum }_{{e}^{′}} \frac{{Mge}^{2} {Mee}^{′2}}{\begin{matrix}\begin{matrix}( Ege - ℏω - iΓ ge ) \\ ( {Ege}^{′} - 2 ℏω - iΓ {ge}^{′} )\end{matrix} \\ ( Ege - ℏω - iΓ ge )\end{matrix}} -\end{matrix} \\ \frac{{Mge}^{4}}{\begin{matrix}\begin{matrix}( Ege - ℏω - iΓ ge ) \\ ( Ege + ℏω + iΓ ge )\end{matrix} \\ ( Ege - ℏω - iΓ ge )\end{matrix}}\end{matrix} ] \qquad Mathematical Formula ( 2 )$$

The most stable structure in the ground state was computed by a DFT method using a B3LYP functional with a 6-31G* basis function, and Mge, Mee′ and Ege were computed based on the result, whereby the value of Imγ was computed. Assuming that the maximum Imγ value obtained by the computation of a quaterphenyl compound where a methoxy group as an electron-donating substituent is substituted on X and Y is 1, the relative value of the maximum Imγ value of a molecule having other substituents and the two-photon absorption maximum wavelength giving the maximum value are shown in Table 3.

TABLE 3
Imaginary Part of Third-Order Polarizability γ when End Substituent
was Varied, and Wavelength Giving Maximum Value Thereof

| | | | | Two-Photon | | |
|---|---|---|---|---|---|---|
| | | | Computed Value | Absorption Maximum | | |
| Computation | | | (Relative Value) | Wavelength in | | |
| Example | X | Y | of Imγ | Computation/nm | Category | |
| 1 | OCH₃ | OCH₃ | 1 | 580 | D · π · D | Comparative |
| | | | | | | Example* |
| 2 | CN | N(CH₃)₂ | 3.2 | 625 | D · π · A | Comparative |
| | | | | | | Example |
| 3 | CN | CO₂CH₃ | 5.3 | 560 | A · π · A | Invention |
| 4 | CN | COPh | 9.2 | 560 | A · π · A | Invention |
| 5 | CN | NO₂ | 8.5 | 680 | A · π · A | Invention |

*Y. Morel, O. Stephan, C. Andraud, and P. L. Baldeck, Synth, Met., 2001, 124, 237, a model compound.

These results suggest that Imγ is small when a methoxy group as an electron-donating group is substituted on X and Y, Imγ increased in the case of a donor-π-acceptor (D-π-A) type molecule where a cyano group as an electron-withdrawing group is substituted on X and a dimethylamino group as an electron-donating group is substituted on Y, and Imγ greatly increases in general in the case of an A-π-A type molecule where an electron-withdrawing substituent is substituted on both X and Y. Since the two-photon absorption cross-sectional area δ is theoretically proportional to the imaginary part of the third-order hyperpolarizability γ, that is, Imγ, it is understood from the computation results thereof that a structure where an electron-withdrawing substituent is substituted on both X and Y is preferred.

### Example 2

### (Preparation of Two-Photon Absorption Recording Material 5)

Two-Photon Absorption Recording Material 5 was prepared according to the following formulation.

| | | |
|---|---|---|
| Two-Photon absorption compound: D-1 | 7.5 | parts by mass |
| Dye precursor: Lo-11 | 2.1 | parts by mass |
| Binder: polyvinyl acetate (produced by | 500 | parts by mass |
| Aldrich, Mw = 113,000) | | |
| Coating solvent: dichloromethane | 14,400 | parts by mass |

### (Preparation of Two-Photon Absorption Recording Material 6)

Two-Photon Absorption Recording Material 6 was prepared according to the following formulation.

| | | |
|---|---|---|
| Two-Photon absorption compound: D-27 | 62.0 | parts by mass |
| Dye precursor: Lo-11 | 2.1 | parts by mass |
| Binder: polyvinyl acetate (produced by | 500 | parts by mass |
| Aldrich, Mw = 113,000) | | |
| Coating solvent: dichloromethane | 14,400 | parts by mass |

### <Production of Two-Photon Absorption Recording Mediums 5 and 6>

Each of Two-Photon Absorption Recording Materials 5 and 6 prepared above was spin-coated on a slide glass to produce Two-Photon Absorption Recording Mediums 5 and 6 composed of one recording layer having a coating thickness of 1 μm after drying.

<<Evaluation of Sensitivity to Two-Photon Recording (Change of Fluorescent Intensity) with Recording Light at 522 nm>>

### <Evaluation System of Two-Photon Recording/Readout by Fluorescent Intensity Change of Two-Photon Absorption Recording Material>

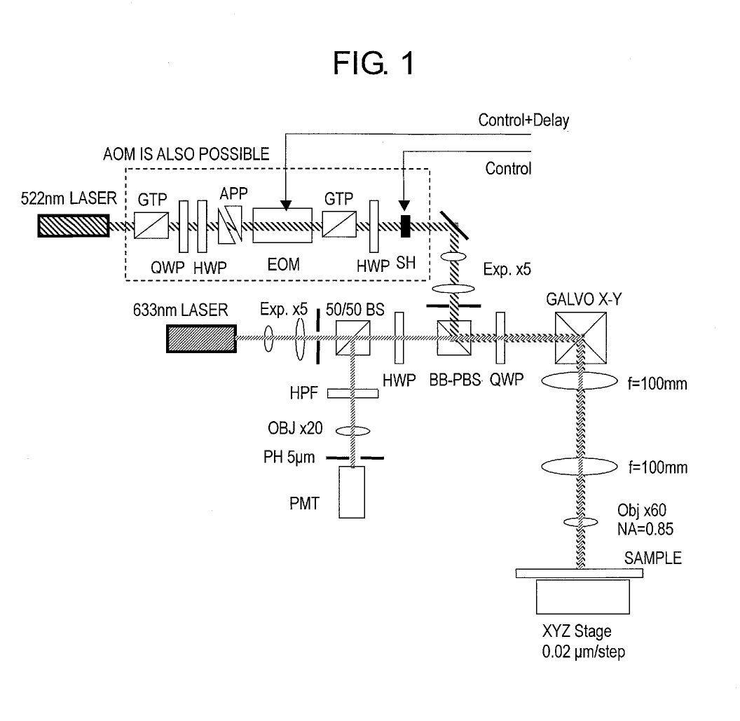

As for the evaluation system of two-photon recording/read out (sometimes simply referred to as “recording/readout”) by fluorescent intensity change of the two-photon absorption recording material, a system shown in FIG. 1 was formulated and used. FIG. 1 is a block diagram schematically showing the recording/readout optical system.

Explanation of the denotations by alphabetical letters in FIG. 1 is as follows.

EOM: electro-optical modulation element, PMT: photomultiplier tube, GAOLVO: galvanometer minor, GTP: Glan-Thompson prism, QWP: ¼ wavelength plate, HWP: ½ wavelength plate, Exp.: beam expander, BS: beam splitter, BB-PBS: broad band polarizing beam splitter, SH: shutter, PH: pinhole, and HPF: high pass filter.

In the two-photon recording, an ultrashort pulsed laser having a wavelength of 522 nm, a pulse width of 500 fs and a repetition frequency of 3 GHz was used and laser light was irradiated on the two-photon recording medium by changing the power and irradiation time of the irradiation laser light according to the sensitivity of the recording medium, thereby performing two-photon recording. In the readout of the recording information, CW light of a He—Ne laser at a wavelength of 633 nm was irradiated while sweeping the inside of the recording layer plane by using a galvanometer minor. A fluorescence signal is observed from a recording pit formed through two-photon absorption by irradiation of light at 633 nm. The fluorescence from a recording pit was received by separating the excitation light of 633 nm and the fluorescence signal and its intensity was evaluated using a photomultiplier tube.

Two-photon recording was performed by irradiating recording light with an average power of 9 mW, and out of the conditions allowing formation of a pit and observation of a fluorescence signal, a shortest recording light irradiation time is defined as the two-photon recording sensitivity of the recording medium. Incidentally, the recording sensitivity was expressed by the time for which the pulsed light irradiated during the opening of the shutter is actually glowing and calculated by shutter opening time×repetition frequency×pulse width.

### <Evaluation of Two-Photon Absorption Recording Medium 5>

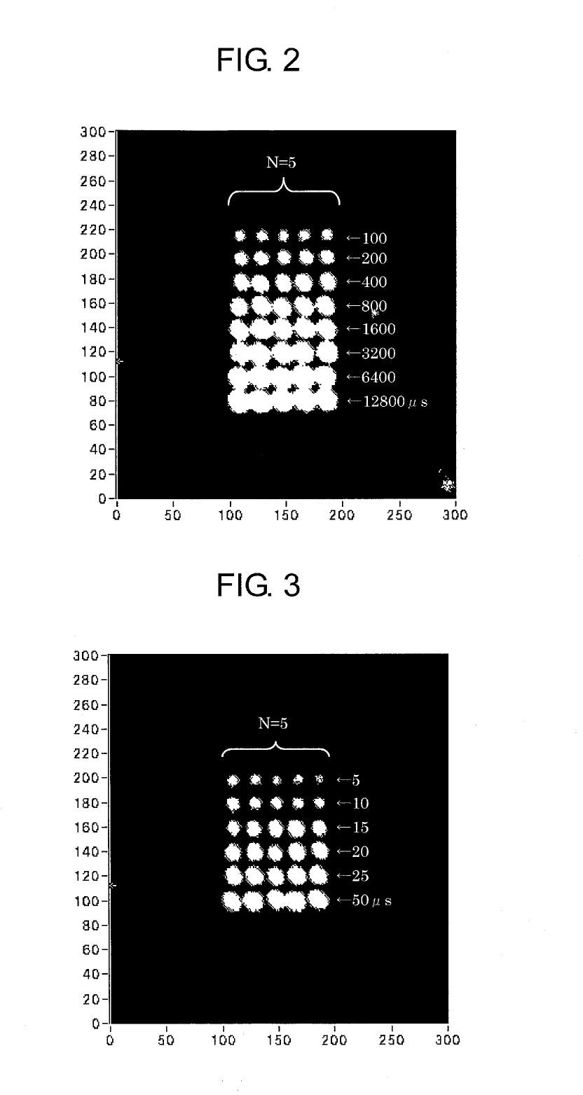

Two-photon recording light with an average power of 9 mW was irradiated on Two-Photon Absorption Recording Medium 5 produced above by varying the shutter opening time to 100, 200, 400, 800, 1,600, 3,200, 6,400 and 12,800 μs (microseconds), thereby performing two-photon recording. The recording was performed 5 times under the same condition (N=5), and FIG. 2 shows an image of fluorescence signals obtained from recording pits. Incidentally, a fluorescent signal could not be observed under the condition of the shutter opening time being shorter than 100 μs.

### <Evaluation of Two-Photon Absorption Recording Medium 6>

Two-photon recording light with an average power of 9 mW was irradiated on Two-Photon Absorption Recording Medium 6 produced above by varying the shutter opening time to 5, 10, 15, 20, 25 and 50 μs (microseconds), thereby performing two-photon recording. The recording was performed 5 times under the same condition (N=5), and FIG. 3 shows an image of fluorescence signals obtained from recording pits. Incidentally, a fluorescent signal could not be observed under the condition of the shutter opening time being shorter than 5 μs.

### <Evaluation of Two-Photon Recording Sensitivity>

The two-photon recording sensitivity was computed from the results obtained and shown in Table 4 below.

TABLE 4

| | Recording Light | Shutter | Two-Photon |
|---|---|---|---|
| Recording | Intensity | Opening | Recording |
| Medium | Dependency | Time | Sensitivity |
| 5 | quadratic | 100 μsec | 150 nsec |
| 6 | quadratic | 5 μsec | 7.5 nsec |

<<Evaluation of Sensitivity to Two-Photon Recording (Change of Fluorescent Intensity) with Recording Light at 405 nm>>

In the recording/readout evaluation optical system shown in FIG. 1, the laser used as the light source for two-photon absorption recording was changed from 522 nm to 405 nm (repetition frequency: 8 MHz, pulse width: 200 fs) that is a second harmonic wave of 810 rim of a Ti:sapphire laser, and the two-photon absorption recording/readout with recording light of 405 nm of Two-Photon Absorption Recording Medium 5 produced above was evaluated.

Laser light was irradiated on Two-Photon Absorption Recording Medium 5 by fixing the shutter opening time to 16 ms (milliseconds) and varying the two-photon absorption recording light intensity in terms of the average power to 25, 50, 100 and 200 μW (microwatt), thereby performing two-photon recording. In the readout of the recording signal, CW light of a He—Ne laser at 633 nm was used and the fluorescence signal generated from the recording pit formed at the recording was detected by the irradiation of reflected light by means of a photomultiplier tube. The obtained signal intensity was evaluated and shown in Table 5 below.

TABLE 5

| Recording Light Irradiation Intensity | Fluorescence Signal Readout |
|---|---|
| (average power) | Intensity (nW) |
| 25 μW | 0.5 |
| 50 μW | 1.5 |
| 100 μW | 6.5 |
| 200 μW | 24.5 |

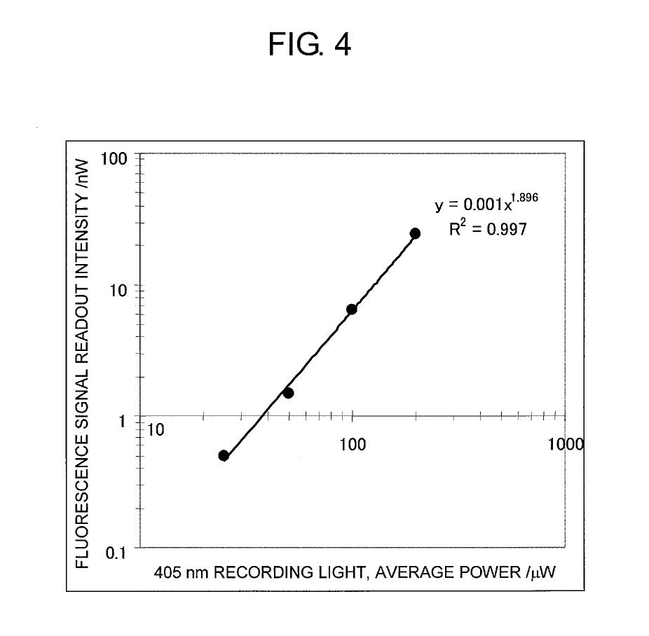

In FIG. 4, the results obtained are plotted. It can be confirmed from FIG. 4 that since the fluorescence signal readout intensity is proportional to the power 1.9 (almost the square) of the recording light intensity at 405 nm, the recording pit is formed by two-photon absorption.

According to the constructions of the two-photon absorption recording material of the present invention, non-resonant two-photon absorption recording can be performed using recording light in a wavelength region shorter than 700 nm and at the same time, sufficient recording/readout properties can be endowed. Also, the two-photon absorption compound of the present invention can exhibit non-resonant two-photon absorption properties with light in a wavelength region shorter than 700 nm and can have a high two-photon absorption cross-sectional area.

The entire disclosure of Japanese Patent Application No. 2008-255808 filed on Sep. 30, 2008 is incorporated herein by reference, as if fully set forth.

What is claimed is:

1. A non-resonant two-photon absorption recording material comprising:
(a) a non-resonant two-photon absorption compound; and
(b) a recording component capable of changing at least either one of a refractive index and a fluorescent intensity,
wherein
the non-resonant two-photon absorption compound (a) is represented by the following formula (1): 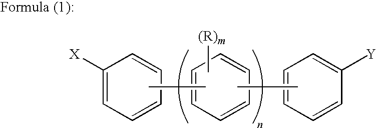
wherein
both of X and Y represent a substituent having a Hammett sigma para value (σp value) of 0 or more and they may be the same as or different from each other;
n represents an integer of 1 to 4;
R represents a substituent and each R may be the same as or different from every other R; and
m represents an integer of 0 to 4.

2. The non-resonant two-photon absorption recording material as claimed in claim 1, wherein
the non-resonant two-photon absorption compound (a) is represented by the following formula (2): 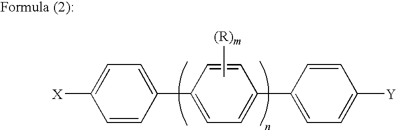
wherein
both of X and Y represent a substituent having a Hammett sigma para value (σp value) of 0 or more and they may be the same as or different from each other;
n represents an integer of 1 to 4;
R represents a substituent and each R may be the same as or different from every other R; and
m represents an integer of 0 to 4.

3. The non-resonant two-photon absorption recording material as claimed in claim 1, wherein
the non-resonant two-photon absorption compound (a) is represented by the following formula (3):

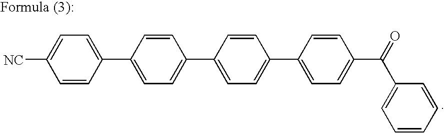

4. The non-resonant two-photon absorption recording material as claimed in claim 1, wherein
the non-resonant two-photon absorption compound (a) is represented by the following formula (4):

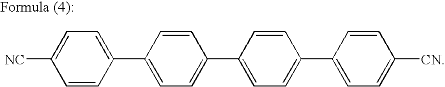

5. A compound represented by the following formula (3): [chemical structure]
